(19) United States
(12) Patent Application Publication
(10) Pub. No.: US 2026/0209159 A1
(43) Pub. Date: Jul. 23, 2026

(54) METHOD FOR PRODUCING POLYCARBOXYLIC ACID ESTER COMPOUND

(71) Applicant: HONSHU CHEMICAL INDUSTRY CO., LTD., Chuo-ku, Tokyo (JP)

(72) Inventors: Rie FUJIOKA, Wakayama-shi (JP); Masato KODERA, Wakayama-shi (JP)

(21) Appl. No.: 19/142,086

(22) PCT Filed: Mar. 25, 2024
(86) PCT No.: PCT/JP2024/011613

§ 371 Date: Jun. 20, 2025

(30) Foreign Application Priority Data
Apr. 11, 2023 (JP) 2023-063921

(51) Int. Cl. C07C 67/02

(52) U.S. Cl. CPC C07C 67/02

(57) ABSTRACT

An object is to provide a method for producing a polycarboxylic acid ester compound in which the content of a specific impurity is reduced and which has improved purity. As a solution, a method for producing a polycarboxylic acid ester compound (1) represented by general formula (1), the method including a step of reaction with an aliphatic alcohol (4), the step including reacting at least one selected from the group consisting of a polycarboxylic acid ester compound (2) represented by general formula (2) and a polycarboxylic acid ester compound (3) represented by general formula (3) with the aliphatic alcohol (4) represented by general formula (4) to obtain the polycarboxylic acid ester compound (1) represented by general formula (1), is provided.

### TECHNICAL FIELD

The present invention relates to a method for producing a polycarboxylic acid ester compound. Particularly, the present invention relates to a method for producing a polycarboxylic acid ester compound in which the content of a specific impurity is reduced and which has improved purity.

### BACKGROUND ART

Polycarboxylic acid compounds are used as raw materials for polyamides, raw materials for allyl ester compounds, and additives such as plasticizers and curing agents, and dicarboxylic acid compounds obtained using bisphenol compounds as raw materials are also known (PTLs 1, 2, etc.).

In recent years, in the fields of applications of such materials, the level of the demand for improvements in various performance capabilities is becoming higher and higher, and raw materials used for the materials are required to have further improved quality so that desired characteristics are exhibited.

One known method for producing a dicarboxylic acid compound obtained using a bisphenol compound as a raw material is to perform an etherification reaction using the bisphenol compound and a halocarboxylic acid or an ester thereof as raw materials to synthesize a dicarboxylic acid or an ester compound thereof. As a method of an etherification reaction for obtaining a dicarboxylic acid compound having improved quality, it is known that, for example, when the etherification reaction is carried out in a state where the amount of water is increased focusing on water remaining in a reaction vessel, a decrease in the yield of the target compound occurs due to a slowdown of the reaction rate and an increase in impurities (PTL 3).

### CITATION LIST

### Patent Literature

PTL 1: Japanese Unexamined Patent Application Publication No. 62-292819
PTL 2: Japanese Unexamined Patent Application Publication No. 05-170702
PTL 3: International Publication No. 2021/054309

### SUMMARY OF INVENTION

### Technical Problem

While the above production method is known, the present inventors have conducted intensive studies so that a dicarboxylic acid ester compound obtained using a bisphenol compound as a raw material has further improved quality, and have found a problem that in the step of an etherification reaction using a bisphenol compound and a halocarboxylic acid ester as raw materials, a by-product resulting from excessive reaction of the halocarboxylic acid ester is formed. In addition, the present inventors have also found a problem that the halocarboxylic acid ester used as a raw material contains, as an impurity, a halocarboxylic acid ester having a different ester group, and this impurity undergoes an etherification reaction to form a by-product having an ester group different from that of the target dicarboxylic acid ester compound.

That is, the present inventors have found a problem that the by-product thus formed hinders the improvement of the purity of the target dicarboxylic acid ester compound. One example of a method for reducing such a by-product is a purification method involving a crystallization operation and a recrystallization operation, but it has been revealed that the purification method involving a recrystallization operation cannot sufficiently reduce such an impurity as in Comparative Examples described later. If the target polycarboxylic acid ester compound contains such an impurity having a different substituent, a failure may occur during the production of a material obtained using this compound, or the physical properties of the material may be adversely affected.

In view of the foregoing problems found by the present inventors, an object of the present invention is to provide a method for producing a polycarboxylic acid ester compound in which the content of a specific impurity is reduced and which has further improved purity.

### Solution to Problem

The present inventors have conducted intensive studies on different methods for reducing such a by-product and found that a reaction with an alcohol having an alkoxy group corresponding to the ester group of the target dicarboxylic acid ester compound can achieve a sufficient reduction, thereby completing the present invention.

The present invention is as follows.
1. A method for producing a polycarboxylic acid ester compound (1) represented by general formula (1), the method including a step of reaction with an aliphatic alcohol (4), the step including reacting at least one selected from the group consisting of a polycarboxylic acid ester compound (2) represented by general formula (2) and a polycarboxylic acid ester compound (3) represented by general formula (3) with the aliphatic alcohol (4) represented by general formula (4) to obtain the carboxylic acid ester compound (1) represented by general formula (1).

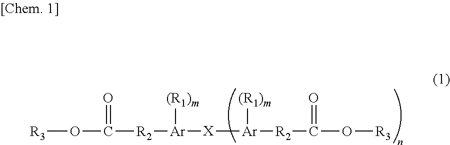

(In formula (1), each Ar independently represents a “(2+m)-valent” monooxy aromatic hydrocarbon group having 6 to 20 carbon atoms, each R₁ independently represents a linear or branched alkyl group having 1 to 6 carbon atoms or a cyclic alkyl group having 5 to 6 carbon atoms, each R₂ independently represents a linear or branched alkylene group having 1 to 4 carbon atoms, R₃ represents a linear or branched alkyl group having 1 to 10 carbon atoms or an alkenyl group having 2 to 10 carbon atoms, each m independently represents 0, 1, or 2, n represents 1 or 2, and X represents a single bond, an oxygen atom, a sulfur atom, a sulfonyl group, a carbonyl group, a divalent group represented by general formula (1a), (1b), or (1c), or a trivalent group represented by general formula (1d) or (1e). In general formula (1), an oxygen atom of Ar is bonded to an aromatic hydrocarbon group included in Ar and to R₂.)

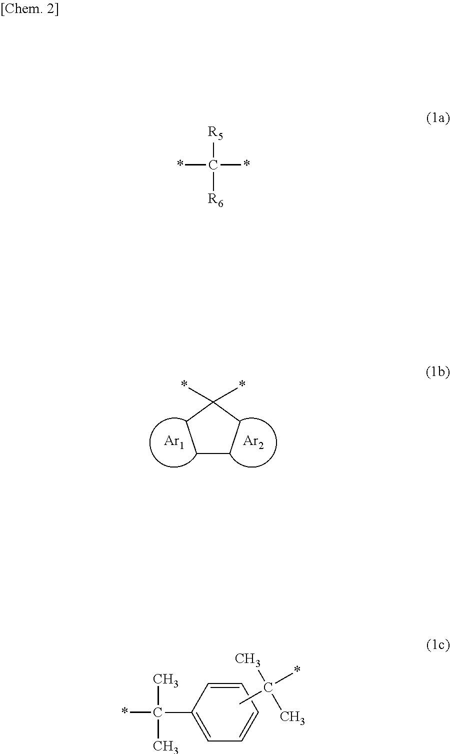

(In general formula (1a), R₅ and R₆ each independently represent a hydrogen atom, an alkyl group having 1 to 10 carbon atoms, an alkyl halide group having 1 to 10 carbon atoms, or an aryl group having 6 to 12 carbon atoms, R₅ and R₆ may be bonded to each other to together form a cycloalkylidene group having 5 to 20 carbon atoms, and in general formula (1b), each Ar independently represents an aryl group having 6 to 12 carbon atoms. Each * in general formulas (1a), (1b), and (1c) represents a bonding position.)

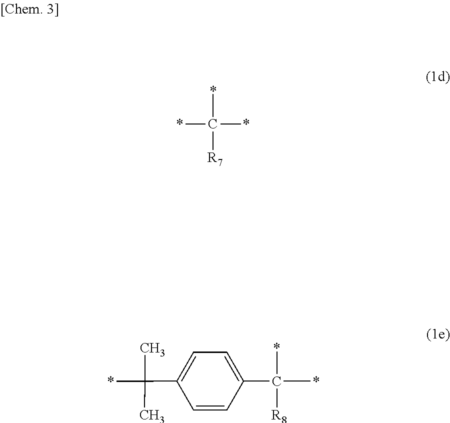

(In general formula (1d), R₇ represents a hydrogen atom, an alkyl group having 1 to 10 carbon atoms, or an aryl group having 6 to 12 carbon atoms. In general formula (1e), R₈ represents a hydrogen atom or an alkyl group having 1 to 10 carbon atoms. Each * in general formulas (1d) and (1e) represents a bonding position.)

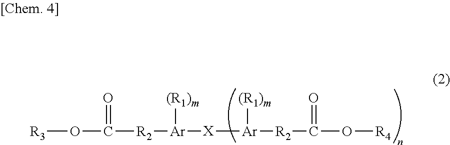

(Ar, R₁, R₂, R₃, m, n, and X in general formula (2) are as defined in general formula (1), and each R₄ independently represents a linear or branched alkyl group having 1 to 10 carbon atoms or an alkenyl group having 2 to 10 carbon atoms that is different from R₃.)

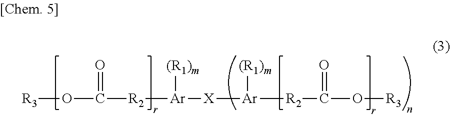

(Ar, R₁, R₂, m, n, X, and R₃ in general formula (3) are as defined in general formula (1), and each r independently represents an integer of 1 to 4. At least one r is 2, 3, or 4.)

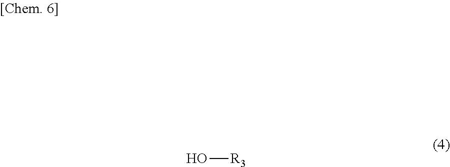

(R₃ in general formula (4) is as defined in general formula (1).)
2. The production method according to 1., wherein the carboxylic acid ester compound (2) represented by general formula (2) and the carboxylic acid ester compound (3) represented by general formula (3) are compounds obtained through an etherification reaction step of performing an etherification reaction using a polyhydroxy aromatic compound (5) represented by general formula (5) and a halocarboxylic acid ester compound composition including a halocarboxylic acid ester compound (6) represented by general formula (6) and a halocarboxylic acid ester compound (7) represented by general formula (7).

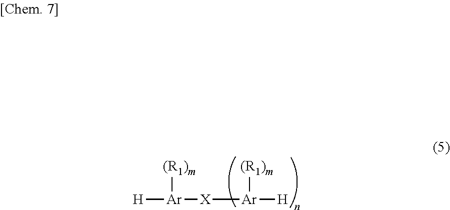

(Ar, R₁, m, n, and X in general formula (5) are as defined in general formula (1). In general formula (5), an oxygen atom of Ar is bonded to an aromatic hydrocarbon group included in Ar and to a hydrogen atom (H) shown in general formula (5).)

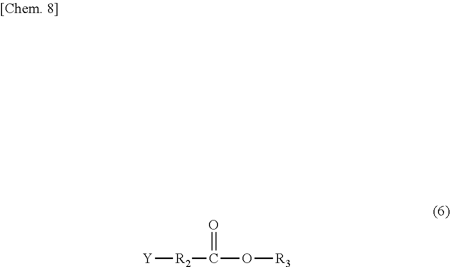

(R₂ and R₃ in general formula (6) are as defined in general formula (1), and Y represents a halogen atom.)

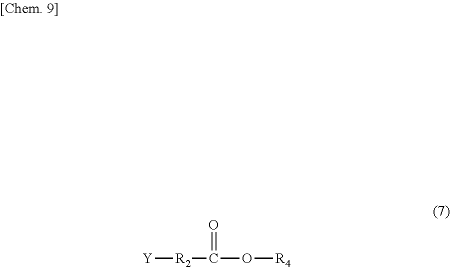

(R₂ and R₄ in general formula (7) are as defined in general formula (2), and Y is as defined in general formula (6).)
3. The production method according to 1., wherein the polycarboxylic acid ester compound (2) is a compound obtained through an etherification reaction step of performing an etherification reaction using a polyhydroxy aromatic compound (5) represented by general formula (5) and a halocarboxylic acid ester compound composition including a halocarboxylic acid ester compound (6) represented by general formula (6) and a halocarboxylic acid ester compound (7) represented by general formula (7).

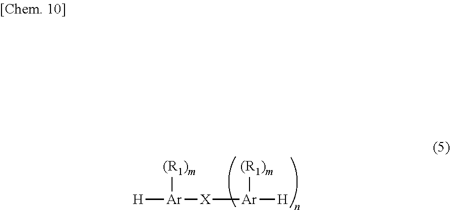

(Ar, R₁, m, n, and X in general formula (5) are as defined in general formula (1). In general formula (5), an oxygen atom of Ar is bonded to an aromatic hydrocarbon group included in Ar and to a hydrogen atom (H) shown in general formula (5).)

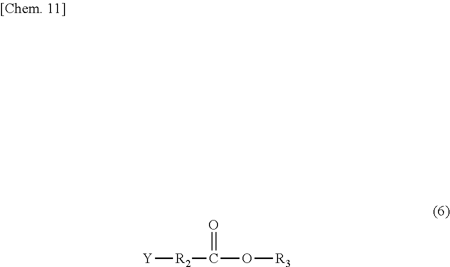

(R₂ and R₃ in general formula (6) are as defined in general formula (1), and Y represents a halogen atom.)

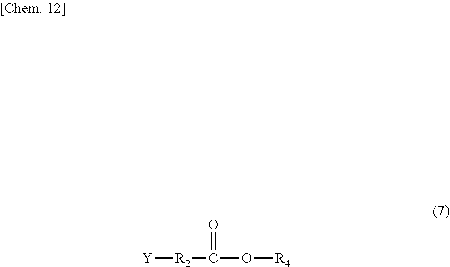

(R₂ and R₄ in general formula (7) are as defined in general formula (2), and Y is as defined in general formula (6).)
4. The production method according to 1., wherein the polycarboxylic acid ester compound (3) is a compound obtained through an etherification reaction step of performing an etherification reaction using a polyhydroxy aromatic compound (5) represented by general formula (5) and a halocarboxylic acid ester compound (6) represented by general formula (6).

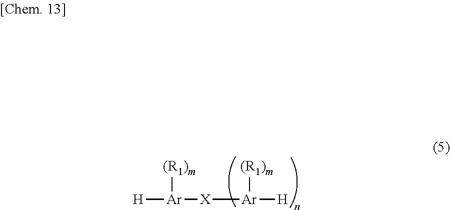

(Ar, R₁, m, n, and X in general formula (5) are as defined in general formula (1). In general formula (5), an oxygen atom of Ar is bonded to an aromatic hydrocarbon group included in Ar and to a hydrogen atom (H) shown in general formula (5).)

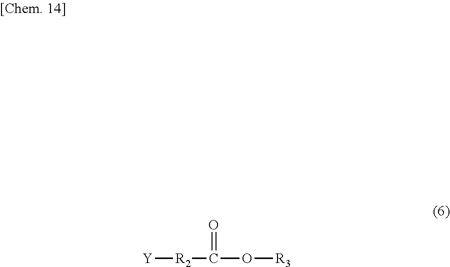

(R₂ and R₃ in general formula (6) are as defined in general formula (1), and Y represents a halogen atom.)
5. The production method according to any one of 1. to 4., wherein Ar is any one group selected from the group consisting of a 1-oxybenzen-4-yl group, a 1-oxybenzen-3-yl group, a 1-oxybenzen-2-yl group, a 1-oxynaphthalen-2-yl group, a 1-oxynaphthalen-4-yl group, a 1-oxynaphthalen-5-yl group, a 2-oxynaphthalen-1-yl group, a 2-oxynaphthalen-6-yl group, a 2-oxynaphthalen-7-yl group, a 4-oxy-3-phenylbenzen-1-yl group, a 9-oxyphenanthren-3-yl group, a 10-oxyphenanthren-9-yl group, a 2-oxyanthracen-7-yl group, a 1-oxy-3-phenylnaphthalen-4-yl group, a 1-oxy-3-phenylnaphthalen-5-yl group, a 2-oxy-1-phenylnaphthalen-6-yl group, a 2-oxy-1-phenylnaphthalen-7-yl group, a 2-oxy-3-phenylnaphthalen-6-yl group, a 2-oxy-3-phenylnaphthalen-7-yl group, a 4-oxy-3-(1-naphthyl)benzen-1-yl group, a 4-oxy-3-(2-naphthyl)benzen-1-yl group, a 4-oxy-3,5-diphenylbenzen-1-yl group, a 4-oxy-2-phenylphenanthren-6-yl group, a 4-oxy-2-phenylphenanthren-7-yl group, a 4-oxy-2-phenylphenanthren-8-yl group, a 4-oxy-2-phenylphenanthren-9-yl group, a 4-oxy-2-phenylphenanthren-10-yl group, and a 2-oxy-3-phenylanthracen-7-yl group.
6. The production method according to 5., wherein n is 2, and X is a direct bond.
7. The production method according to 6., wherein R₃ is a linear or branched alkyl group having 1 to 10 carbon atoms.

### Advantageous Effects of Invention

According to the production method of the present invention, a specific impurity that is contained in the polycarboxylic acid ester compound (1) and that cannot be removed or can only be removed with limited effectiveness by a crystallization operation or a recrystallization operation can be reduced, and thus the polycarboxylic acid ester compound (1) having improved quality can be produced.

### DESCRIPTION OF EMBODIMENTS

### <Production Method of Present Invention>

A method for producing a polycarboxylic acid ester compound (1) represented by general formula (1) according to the present invention includes a step of reaction with an aliphatic alcohol (4), the step including reacting at least one selected from the group consisting of a polycarboxylic acid ester compound (2) represented by general formula (2) and a polycarboxylic acid ester compound (3) represented by general formula (3) with the aliphatic alcohol (4) represented by general formula (4) to obtain the carboxylic acid ester compound (1) represented by general formula (1).

In one embodiment, the step of reaction with an aliphatic alcohol (4) includes reacting a polycarboxylic acid ester compound (2) with the aliphatic alcohol (4) to obtain the carboxylic acid ester compound (1); in one embodiment, the step of reaction with an aliphatic alcohol (4) includes reacting a polycarboxylic acid ester compound (3) with the aliphatic alcohol (4) to obtain the carboxylic acid ester compound (1); and in one embodiment, the step of reaction with an aliphatic alcohol (4) includes reacting a polycarboxylic acid ester compound (2) and a polycarboxylic acid ester compound (3) with the aliphatic alcohol (4) to obtain the carboxylic acid ester compound (1).

### <Polycarboxylic Acid Ester Compound (1) Represented by General Formula (1)>

Each Ar in general formula (1) independently represents a “(2+m)-valent” monooxy aromatic hydrocarbon group having 6 to 20 carbon atoms. In general formula (1), an oxygen atom of Ar is bonded to an aromatic hydrocarbon group included in Ar and to R₂.

First, the case where m is 0, that is, a divalent monooxy aromatic hydrocarbon group having 6 to 20 carbon atoms will be described.

Among divalent monooxy aromatic hydrocarbon groups having 6 to 20 carbon atoms, divalent monooxy aromatic hydrocarbon groups having 6 to 16 carbon atoms are preferred, divalent monooxy aromatic hydrocarbon groups having 6 to 14 carbon atoms are more preferred, divalent monooxy aromatic hydrocarbon groups having 6, 10, or 14 carbon atoms are still more preferred, and divalent monooxy aromatic hydrocarbon groups having 10 or 14 carbon atoms are particularly preferred.

Specific examples of divalent monooxy aromatic hydrocarbon groups having 6 carbon atoms include a 1-oxybenzen-4-yl group, a 1-oxybenzen-3-yl group, and a 1-oxybenzen-2-yl group.

Specific examples of divalent monooxy aromatic hydrocarbon groups having 10 carbon atoms include a 1-oxynaphthalen-2-yl group, a 1-oxynaphthalen-4-yl group, a 1-oxynaphthalen-5-yl group, a 2-oxynaphthalen-1-yl group, a 2-oxynaphthalen-6-yl group, and a 2-oxynaphthalen-7-yl group.

One specific example of a divalent monooxy aromatic hydrocarbon group having 12 carbon atoms is a 4-oxy-3-phenylbenzen-1-yl group.

Specific examples of divalent monooxy aromatic hydrocarbon groups having 14 carbon atoms include a 9-oxyphenanthren-3-yl group, a 10-oxyphenanthren-9-yl group, and a 2-oxyanthracen-7-yl group.

Among the divalent monooxy aromatic hydrocarbon groups having 14 carbon atoms, monooxyphenanthrenyl groups are more preferred. Therefore, among them, a 9-oxyphenanthren-3-yl group or a 10-oxyphenanthren-9-yl group is preferred.

Specific examples of divalent monooxy aromatic hydrocarbon groups having 16 carbon atoms include a 1-oxy-3-phenylnaphthalen-4-yl group, a 1-oxy-3-phenylnaphthalen-5-yl group, a 2-oxy-1-phenylnaphthalen-6-yl group, a 2-oxy-1-phenylnaphthalen-7-yl group, a 2-oxy-3-phenylnaphthalen-6-yl group, a 2-oxy-3-phenylnaphthalen-7-yl group, a 4-oxy-3-(1-naphthyl)benzen-1-yl group, and a 4-oxy-3-(2-naphthyl)benzen-1-yl group.

One specific example of a divalent monooxy aromatic hydrocarbon group having 18 carbon atoms is a 4-oxy-3,5-diphenylbenzen-1-yl group.

Specific examples of divalent monooxy aromatic hydrocarbon groups having 20 carbon atoms include a 4-oxy-2-phenylphenanthren-6-yl group, a 4-oxy-2-phenylphenanthren-7-yl group, a 4-oxy-2-phenylphenanthren-8-yl group, a 4-oxy-2-phenylphenanthren-9-yl group, a 4-oxy-2-phenylphenanthren-10-yl group, and a 2-oxy-3-phenylanthracen-7-yl group.

Any one group selected from the group consisting of the above specific examples of divalent monooxy aromatic hydrocarbon groups having 6 to 20 carbon atoms can also be selected. That is, any one group selected from the group consisting of a 1-oxybenzen-4-yl group, a 1-oxybenzen-3-yl group, a 1-oxybenzen-2-yl group, a 1-oxynaphthalen-2-yl group, a 1-oxynaphthalen-4-yl group, a 1-oxynaphthalen-5-yl group, a 2-oxynaphthalen-1-yl group, a 2-oxynaphthalen-6-yl group, a 2-oxynaphthalen-7-yl group, a 4-oxy-3-phenylbenzen-1-yl group, a 9-oxyphenanthren-3-yl group, a 10-oxyphenanthren-9-yl group, a 2-oxyanthracen-7-yl group, a 1-oxy-3-phenylnaphthalen-4-yl group, a 1-oxy-3-phenylnaphthalen-5-yl group, a 2-oxy-1-phenylnaphthalen-6-yl group, a 2-oxy-1-phenylnaphthalen-7-yl group, a 2-oxy-3-phenylnaphthalen-6-yl group, a 2-oxy-3-phenylnaphthalen-7-yl group, a 4-oxy-3-(1-naphthyl)benzen-1-yl group, a 4-oxy-3-(2-naphthyl)benzen-1-yl group, a 4-oxy-3,5-diphenylbenzen-1-yl group, a 4-oxy-2-phenylphenanthren-6-yl group, a 4-oxy-2-phenylphenanthren-7-yl group, a 4-oxy-2-phenylphenanthren-8-yl group, a 4-oxy-2-phenylphenanthren-9-yl group, a 4-oxy-2-phenylphenanthren-10-yl group, and a 2-oxy-3-phenylanthracen-7-yl group can also be selected.

Among them, any one group selected from the group consisting of a 1-oxybenzen-4-yl group, a 2-oxynaphthalen-1-yl group, a 2-oxynaphthalen-6-yl group, a 4-oxy-3-phenylbenzen-1-yl group, a 9-oxyphenanthren-3-yl group, and a 10-oxyphenanthren-9-yl group is preferred, any one group selected from the group consisting of a 1-oxybenzen-4-yl group, a 2-oxynaphthalen-1-yl group, and a 10-oxyphenanthren-9-yl group is more preferred, and a 2-oxynaphthalen-1-yl group or a 10-oxyphenanthren-9-yl group is particularly preferred.

Next, the case where m is 1 or 2, that is, a trivalent or tetravalent monooxy aromatic hydrocarbon group having 6 to 20 carbon atoms is a group in which one or two, which depends on the number of m, of the hydrogen atoms of the aromatic hydrocarbon group moiety in the case where m is 0 are replaced with bonding positions so as to enable bonding to a group represented by R₁.

Each R₁ in general formula (1) independently represents an alkyl group having 1 to 6 carbon atoms or a cyclic alkyl group having 5 to 6 carbon atoms. In particular, R₁ is preferably an alkyl group having 1 to 4 carbon atoms or a cyclohexyl group independently at each occurrence, more preferably a methyl group, a t-butyl group, or a cyclohexyl group independently at each occurrence, particularly preferably a methyl group.

Each R₂ in general formula (1) independently represents a linear or branched alkylene group having 1 to 4 carbon atoms. In particular, R₂ is particularly preferably a methylene group.

R₃ in general formula (1) represents a linear or branched alkyl group having 1 to 10 carbon atoms or an alkenyl group having 2 to 10 carbon atoms. In particular, R₃ is preferably a linear or branched alkyl group having 1 to 10 carbon atoms, more preferably a linear or branched alkyl group having 1 to 6 carbon atoms, still more preferably a methyl group or an ethyl group, particularly preferably an ethyl group.

Each m independently represents 0, 1, or 2. In particular, m is preferably 0 or 1, particularly preferably 0.

In the case where m is 1, a preferred position at which R₁ is bonded is as follows: 1-oxybenzen-4-yl group, 2-position; 2-oxynaphthalen-1-yl group, 6-position; 2-oxynaphthalen-6-yl group, 5-position; 4-oxy-3-phenylbenzen-1-yl group, 5-position; 9-oxyphenanthren-3-yl group, 10-position; 10-oxyphenanthren-9-yl group, 6-position.

In the case where m is 2, preferred positions at which R₁ is bonded is as follows: 1-oxybenzen-4-yl group, 2-position and 5-position.

n represents 1 or 2, preferably 1.

When n is 1, X in general formula (1) is a single bond, an oxygen atom, a sulfur atom, a sulfonyl group, a carbonyl group, or a divalent group represented by general formula (1a), (1b), or (1c). In particular, X is preferably a single bond, a divalent group represented by general formula (1a), a divalent group represented by (1b), or a divalent group represented by (1c), more preferably a single bond, a divalent group represented by (1b), or a divalent group represented by (1c), particularly preferably a single bond.

When n is 2, X in general formula (1) is a trivalent group represented by general formula (1d) or (1e), preferably a trivalent group represented by (1e).

When X in general formula (1) is a divalent group represented by general formula (1a), R₃ and R₄ are each independently more preferably a hydrogen atom, an alkyl group having 1 to 6 carbon atoms, an alkyl halide group having 1 to 6 carbon atoms, or an aryl group having 6 to 12 carbon atoms, still more preferably a hydrogen atom or an alkyl group having 1 to 4 carbon atoms, particularly preferably a hydrogen atom, a methyl group, or an ethyl group.

R₃ and R₄ may be bonded to each other to together form a cycloalkylidene group having 5 to 20 carbon atoms. The cycloalkylidene group having 5 to 20 carbon atoms may include a branched-chain alkyl group. The cycloalkylidene group preferably has 5 to 15 carbon atoms, more preferably has 6 to 12 carbon atoms, and particularly preferably has 6 to 9 carbon atoms.

Specific examples of the cycloalkylidene group include a cyclopentylidene group (5 carbon atoms), a cyclohexylidene group (6 carbon atoms), a 3-methylcyclohexylidene group (7 carbon atoms), a 4-methylcyclohexylidene group (7 carbon atoms), a 3, 3,5-trimethylcyclohexylidene group (9 carbon atoms), a cycloheptylidene group (7 carbon atoms), a bicyclo[2. 2. 1]heptane-2,2-diyl group (7 carbon atoms), a 1, 7, 7-trimethylbicyclo[2.2.1]heptane-2,2-diyl group (10 carbon atoms), a 4, 7, 7-trimethylbicyclo[2.2.1]heptane-2, 2-diyl group (10 carbon atoms), a tricyclo[5. 2. 1. 02, 6]decane-8,8-diyl group (10 carbon atoms), a 2, 2-adamantylidene group (10 carbon atoms), and a cyclododecanylidene group (12 carbon atoms). Preferred are a cyclohexylidene group (6 carbon atoms), a 3-methylcyclohexylidene group (7 carbon atoms), a 4-methylcyclohexylidene group (7 carbon atoms), a 3,3,5-trimethylcyclohexylidene group (9 carbon atoms), and a cyclododecanylidene group (12 carbon atoms), more preferred are a cyclohexylidene group (6 carbon atoms), a 3,3,5-trimethylcyclohexylidene group (9 carbon atoms), and a cyclododecanylidene group (12 carbon atoms), and particularly preferred are a cyclohexylidene group (6 carbon atoms) and a 3, 3, 5-trimethylcyclohexylidene group (9 carbon atoms).

When X in general formula (1) is a divalent group represented by general formula (1b), each Ar₁ is preferably independently a benzene ring or a naphthalene ring, and each Ar₁ is more preferably a benzene ring. For example, when each Ar is a benzene ring, the group represented by general formula (1b) is a fluorenylidene group.

In a preferred form in the case where X in general formula (1) is a divalent group represented by general formula (1c), the divalent group is preferably a divalent group represented by formula (1c′) or formula (1c″).

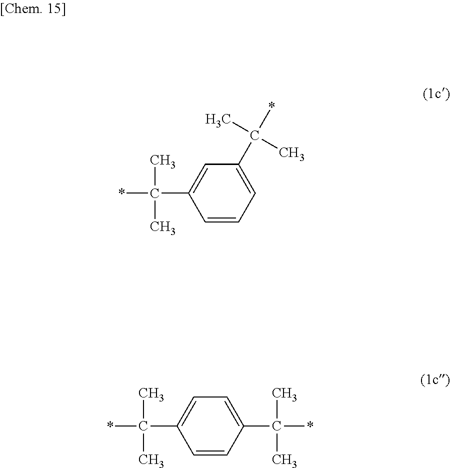

In a preferred form in the case where X in general formula (1) is a trivalent group represented by general formula (1d), R₇ is preferably a hydrogen atom, an alkyl group having 1 to 6 carbon atoms, or a phenyl group, R₇ is more preferably a hydrogen atom, a methyl group, or a phenyl group, and R₇ is particularly preferably a hydrogen atom or a methyl group.

In a preferred form in the case where X in general formula (1) is a trivalent group represented by general formula (1e), R₈ is preferably a hydrogen atom or a methyl group, and Rₑ is particularly preferably a methyl group.

Regarding specific examples of the polycarboxylic acid ester compound (1), specific examples of compounds represented by general formula (1) where each Ar is a 1-oxybenzen-4-yl group include compounds (1-1) to (1-6). [Chem. 16]

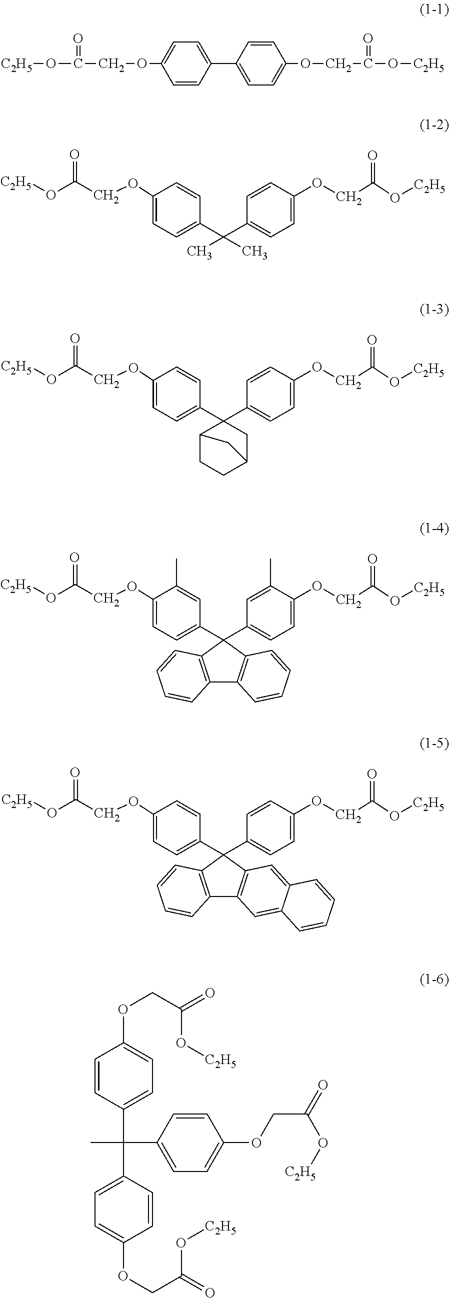

Specific examples of compounds represented by general formula (1) where each Ar is a 2-oxynaphthalen-1-yl group include compounds (1-7) and (1-8).

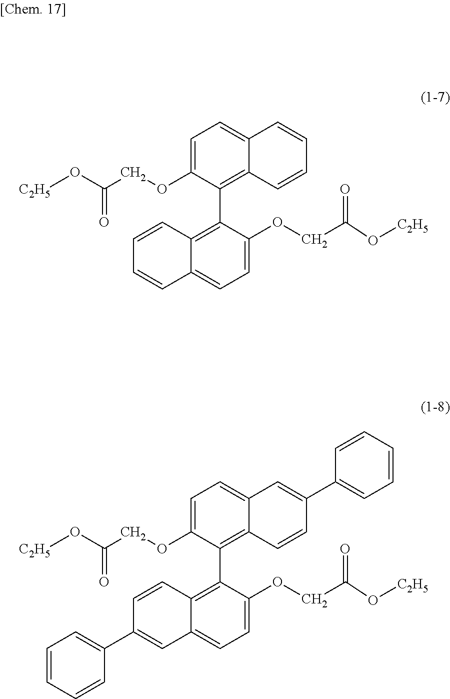

One specific example of a compound represented by general formula (1) where each Ar is a 2-oxynaphthalen-6-yl group is compound (1-9).

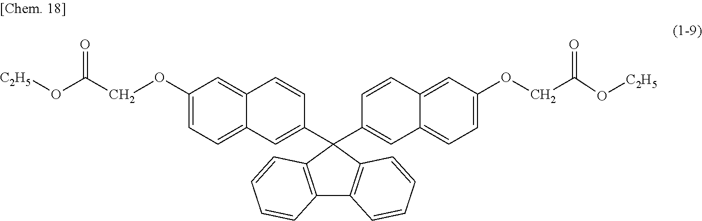

Specific examples of compounds represented by general formula (1) where each Ar is a 4-oxy-3-phenylbenzen-1-yl group include compounds (1-10) and (1-11).

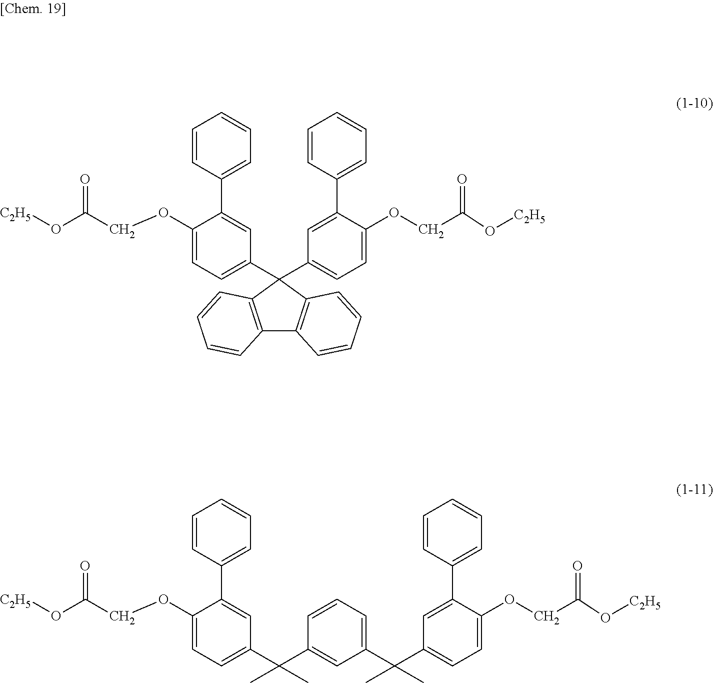

Specific examples of compounds represented by general formula (1) where each Ar is a 10-oxyphenanthren-9-yl group include compounds (1-12) and (1-13).

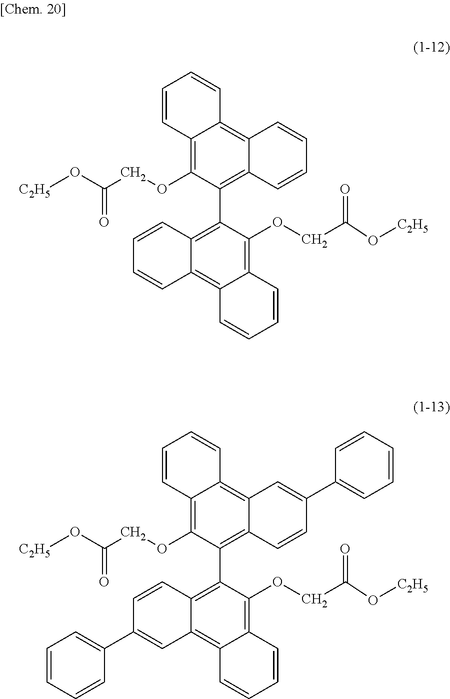

### <Polycarboxylic Acid Ester Compound (2) Represented by General Formula (2)>

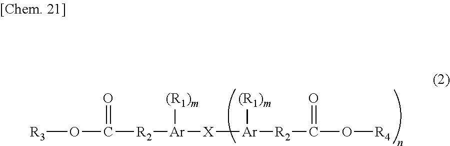

Ar, R₁, R₂, R₃, m, n, and X in general formula (2) are as defined in general formula (1), and specific examples and preferred forms thereof are also the same.

Each R₄ independently represents a linear or branched alkyl group having 1 to 10 carbon atoms or an alkenyl group having 2 to 10 carbon atoms that is different from R₃. In particular, R₄ is preferably a linear or branched alkyl group having 1 to 10 carbon atoms, more preferably a linear or branched alkyl group having 1 to 6 carbon atoms, still more preferably a methyl group or an ethyl group, particularly preferably a methyl group.

Regarding the polycarboxylic acid ester compound (2), specific examples of compounds represented by general formula (2) where each Ar is a 1-oxybenzen-4-yl group, and R₃ and R₄ are an ethyl group and a methyl group include compounds (2-1) to (2-6) and (2-6′).

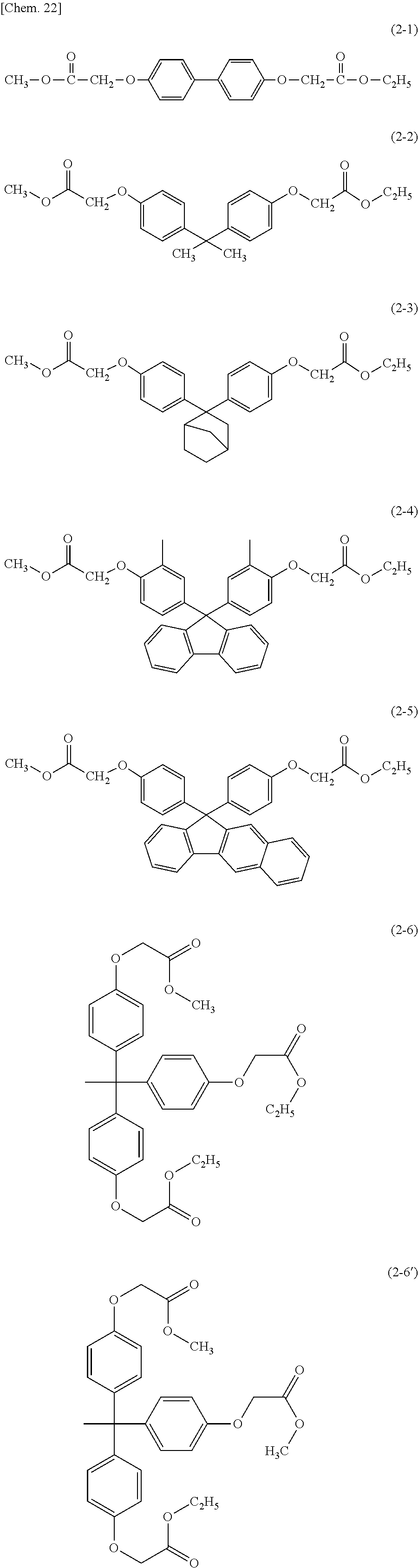

Specific examples of compounds represented by general formula (2) where each Ar is a 2-oxynaphthalen-1-yl group, and R₃ and R₄ are an ethyl group and a methyl group include compounds (2-7) and (2-8).

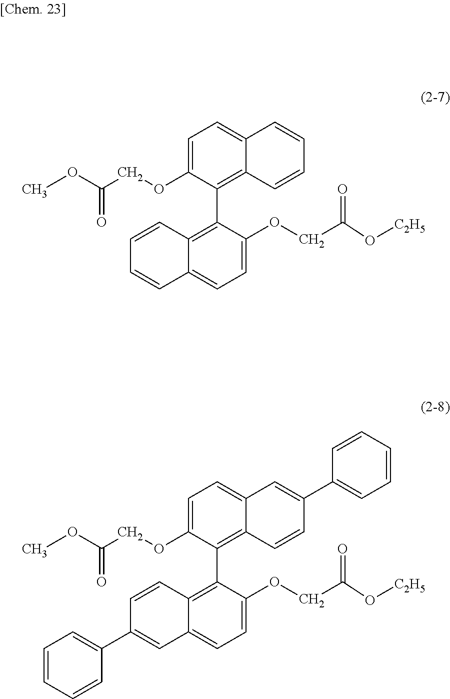

One specific example of a compound represented by general formula (2) where each Ar is a 2-oxynaphthalen-6-yl group, and R₃ and R₄ are an ethyl group and a methyl group is compound (2-9).

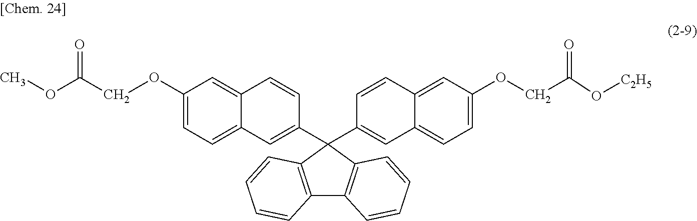

Specific examples of compounds represented by general formula (2) where each Ar is a 4-oxy-3-phenylbenzen-1-yl group, and R₃ and R₄ are an ethyl group and a methyl group include compounds (2-10) and (2-11).

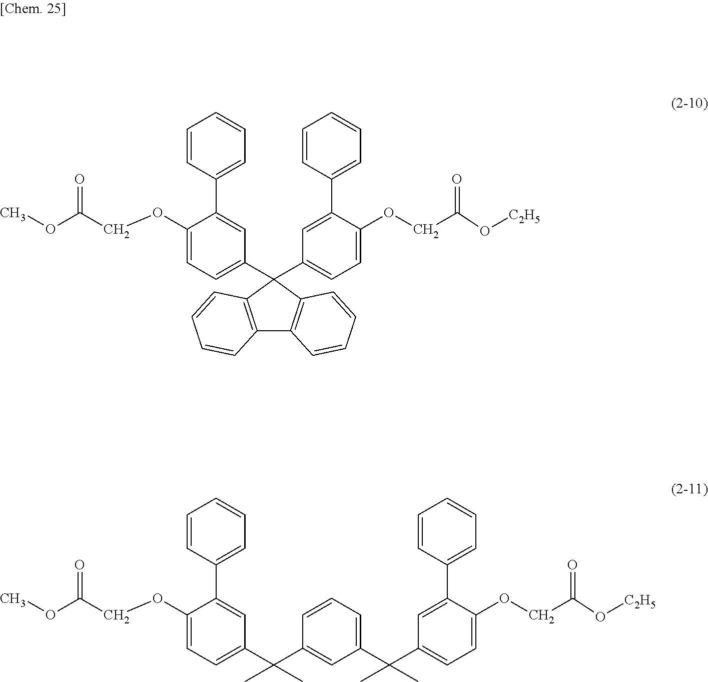

Specific examples of compounds represented by general formula (2) where each Ar is a 10-oxyphenanthren-9-yl group, and R₃ and R₄ are an ethyl group and a methyl group include compounds (2-12) and (2-13).

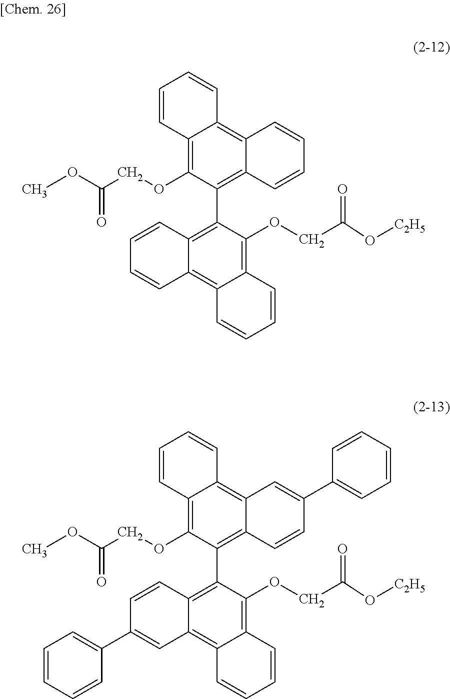

The polycarboxylic acid ester compound (2) used in the production method of the present invention is not limited, and the production method for obtaining it is also not limited. For example, the polycarboxylic acid ester compound (2) can be produced by a method including an etherification reaction step of performing an etherification reaction using a polyhydroxy aromatic compound (5) represented by general formula (5) and a halocarboxylic acid ester compound composition including a halocarboxylic acid ester compound (6) represented by general formula (6) and a halocarboxylic acid ester compound (7) represented by general formula (7), as shown by the following reaction formula. The polycarboxylic acid ester compound (2) is preferably obtained by this method. Through this etherification reaction step, the polycarboxylic acid ester compound (1) may be formed, and is preferably formed.

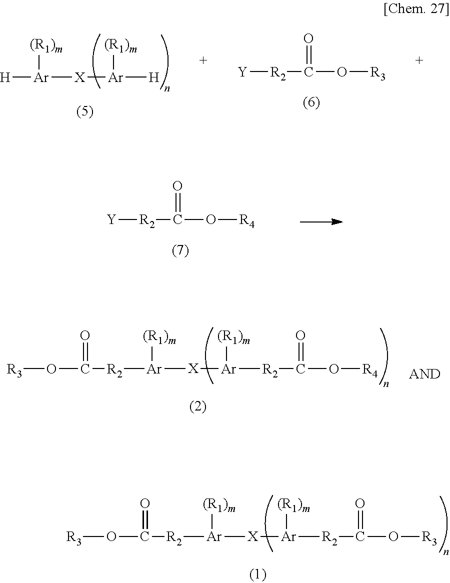

One specific example of such a method is a method in which compound (5-7) as the polyhydroxy aromatic compound (5) and a halocarboxylic acid ester compound composition including ethyl chloroacetate (6-2) as the halocarboxylic acid ester compound (6) and methyl chloroacetate (7-1) as the halocarboxylic acid ester compound (7) are used to produce compound (2-7), as shown by the following reaction formula. Through this etherification reaction step, compound (1-7) may be formed as the polycarboxylic acid ester compound (1) represented by general formula (1), and is preferably formed.

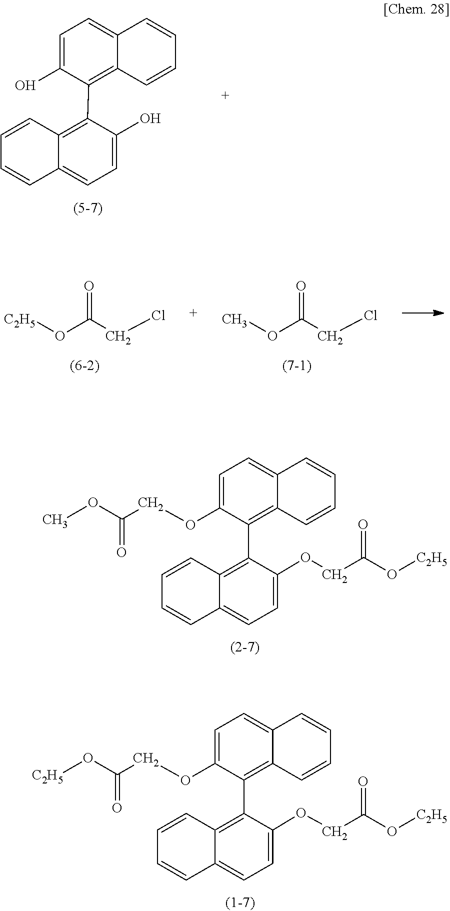

Alternatively, the polycarboxylic acid ester compound (2) can also be produced by carboxylic acid esterification involving reacting a polycarboxylic acid compound in which R₃ and R₄ of the polycarboxylic acid ester compound (2) are both hydrogen atoms with alcohols corresponding to R₃ and R₄. Through this reaction, the polycarboxylic acid ester compound (1) may be formed, and is preferably formed.

One specific example of such a method is a method in which 2, 2′-bis(2-carboxymethoxy)-1,1′-binaphthyl is reacted with ethanol and methanol to produce compound (2-7), as shown by the following reaction formula. Through this reaction, compound (1-7) may be formed as the polycarboxylic acid ester compound (1), and is preferably formed.

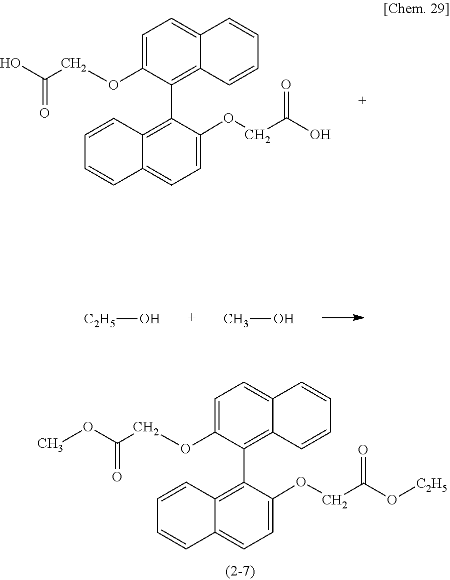

### <Polycarboxylic Acid Ester Compound (3) Represented by General Formula (3)>

Ar, R₁, R₂, m, n, X, and R₃ in general formula (3) are as defined in general formula (1), and specific examples and preferred forms thereof are also the same.

Each r independently represents an integer of 1 to 4. At least one r is 2, 3, or 4.

Regarding the polycarboxylic acid ester compound (3), specific examples of compounds represented by general formula (3) where each Ar is a 1-oxybenzen-4-yl group include compounds (3-1) to (3-6) and (3-6′).

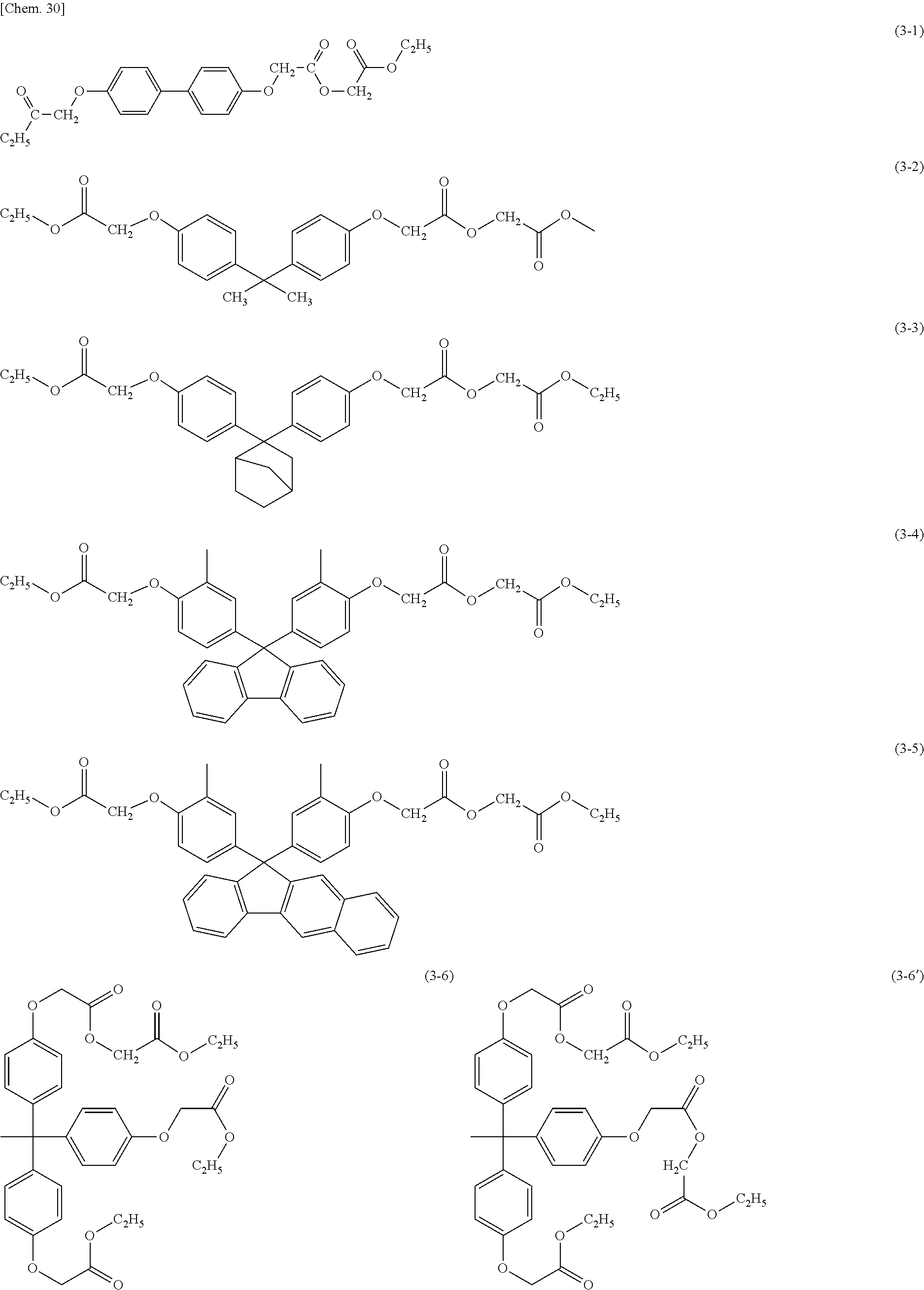

Specific examples of compounds represented by general formula (3) where each Ar is a 2-oxynaphthalen-1-yl group include compounds (3-7) and (3-8).

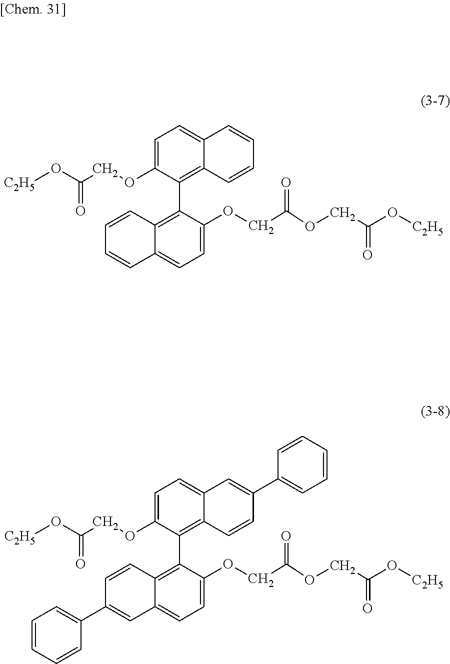

One specific example of a compound represented by general formula (3) where each Ar is a 2-oxynaphthalen-6-yl group is compound (3-9).

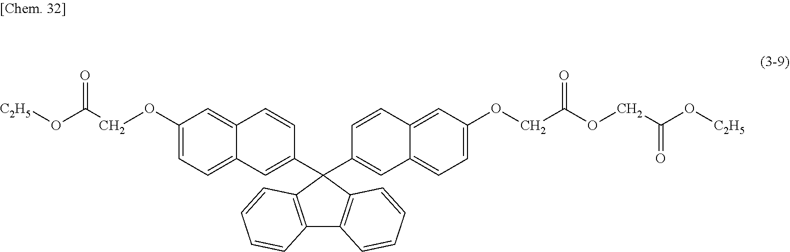

Specific examples of compounds represented by general formula (3) where each Ar is a 4-oxy-3-phenylbenzen-1-yl group include compounds (3-10) and (3-11).

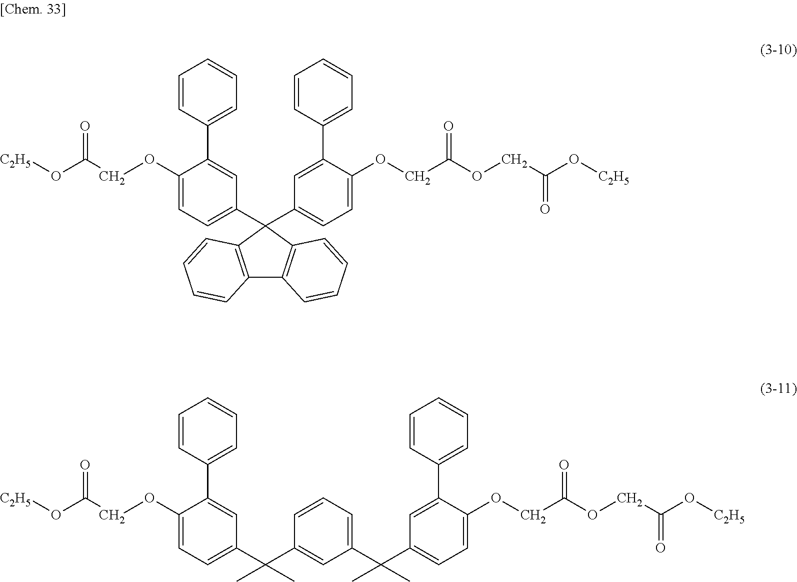

Specific examples of compounds represented by general formula (3) where each Ar is a 10-oxyphenanthren-9-yl group include compounds (3-12) and (3-13).

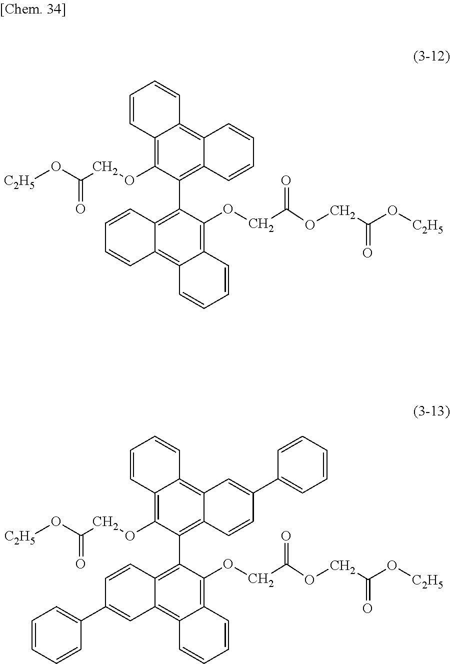

The polycarboxylic acid ester compound (3) used in the production method of the present invention is not limited, and the production method for obtaining it is also not limited. For example, the polycarboxylic acid ester compound (3) can be produced by a method including an etherification reaction step of performing an etherification reaction using a polyhydroxy aromatic compound (5) and a halocarboxylic acid ester compound (6) or a halocarboxylic acid ester compound composition including a halocarboxylic acid ester compound (6) and a halocarboxylic acid ester compound (7), as shown by the following reaction formula. The polycarboxylic acid ester compound (3) is preferably obtained by this method. Through this etherification reaction step, the polycarboxylic acid ester compound (1) may be formed, and is preferably formed.

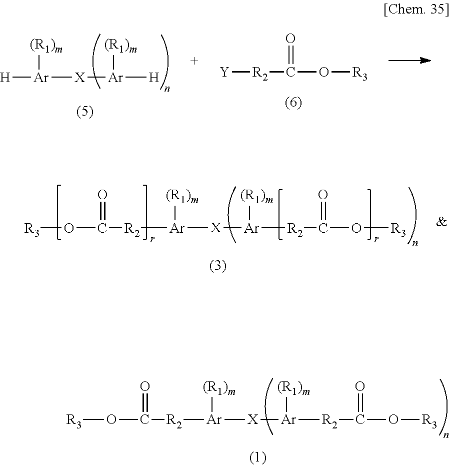

One specific example of such a method is a method in which compound (5-7) as the polyhydroxy aromatic compound (5) and ethyl chloroacetate (6-2) as the halocarboxylic acid ester compound (6) are used to produce compound (3-7), as shown by the following reaction formula. Through this etherification reaction step, compound (1-7) may be formed as the polycarboxylic acid ester compound (1), and is preferably formed.

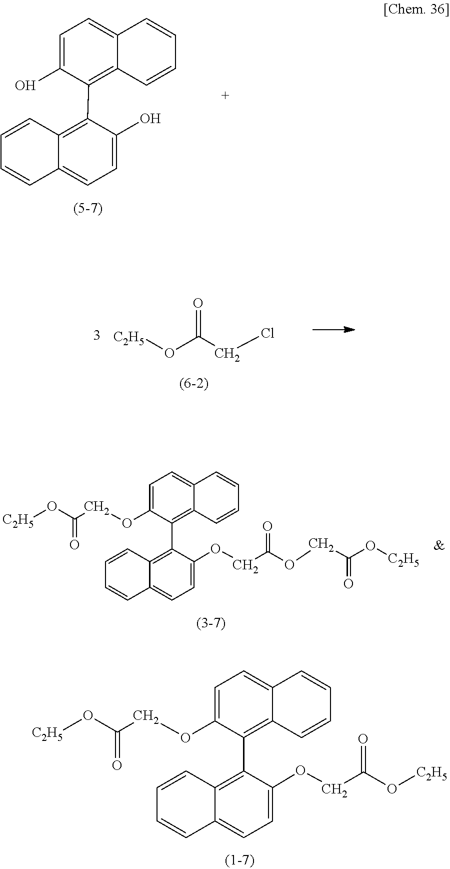 [SMILES: Oc1ccc2ccccc2c1-c1c(O)ccc2ccccc12 ; CCOC(=O)CCl ; CCOC(=O)COC(=O)COc1ccc2ccccc2c1-c1c(OCC(=O)OCC)ccc2ccccc12 ; CCOC(=O)COc1ccc2ccccc2c1-c1c(OCC(=O)OCC)ccc2ccccc12]

In the method for producing a polycarboxylic acid ester compound (1) according to the present invention, at least one selected from the group consisting of a polycarboxylic acid ester compound (2) and a polycarboxylic acid ester compound (3) may include the polycarboxylic acid ester compound (2) alone, the polycarboxylic acid ester compound (3) alone, or both the polycarboxylic acid ester compound (2) and the polycarboxylic acid ester compound (3).

When both the polycarboxylic acid ester compound (2) and the polycarboxylic acid ester compound (3) are included, a compound obtained by a production method involving a method including an etherification reaction step of performing an etherification reaction using a polyhydroxy aromatic compound (5) and a halocarboxylic acid ester compound composition including a halocarboxylic acid ester compound (6) and a halocarboxylic acid ester compound (7), as shown by the following reaction formula, among the above-described methods for obtaining the compounds, is preferred. Through this etherification reaction step, the polycarboxylic acid ester compound (1) may be formed, and is preferably formed.

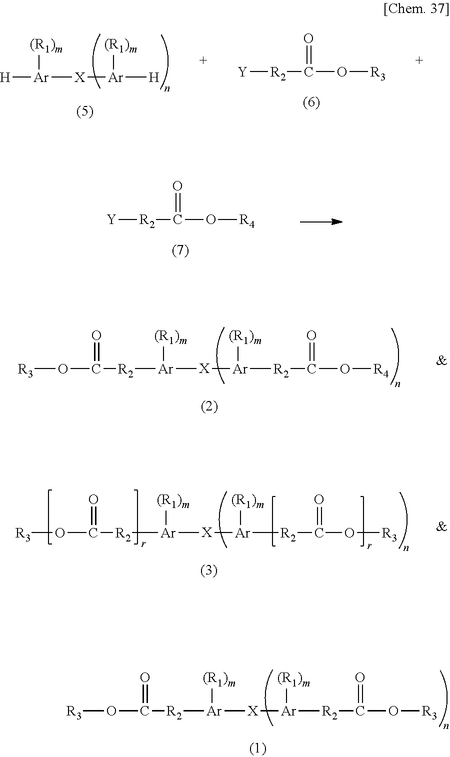

One specific example of such a method is a method in which compound (5-7) given later as the polyhydroxy aromatic compound (5) and a halocarboxylic acid ester compound composition including ethyl chloroacetate (6-2) as the halocarboxylic acid ester compound (6) and methyl chloroacetate (7-1) as the halocarboxylic acid ester compound (7) are used to produce compound (2-7) and compound (3-7), as shown by the following reaction formula. Through this etherification reaction step, compound (1-7) may be formed as the polycarboxylic acid ester compound (1), and is preferably formed.

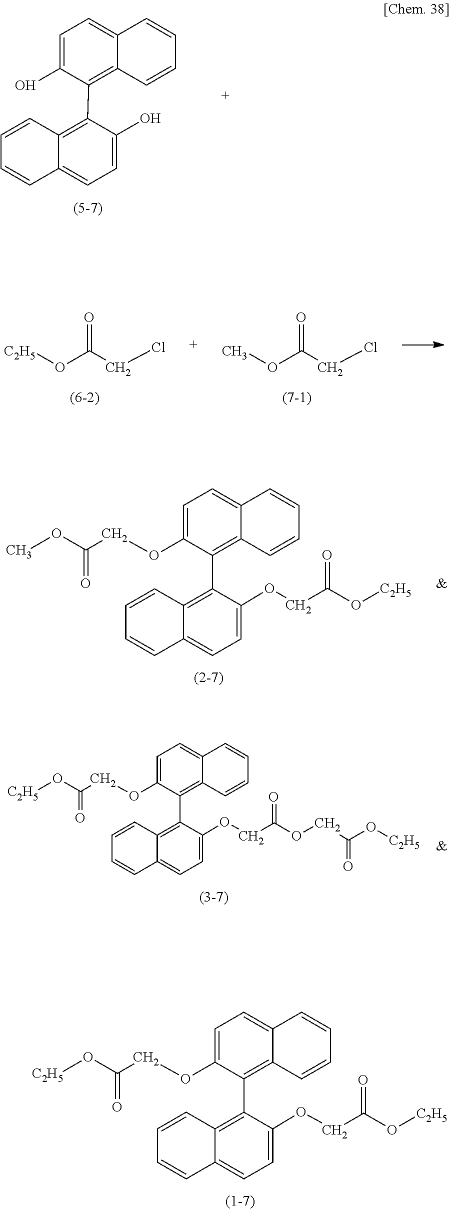

The polycarboxylic acid ester compound (2) and the polycarboxylic acid ester compound (3) may be of only one type or two or more types within the range of the compounds.

### <Polyhydroxy Aromatic Compound (5) Represented by General Formula (5)>

Ar, R₁, m, n, and X in general formula (5) are as defined in general formula (1), and specific examples and preferred forms thereof are also the same.

In general formula (5), an oxygen atom of Ar is bonded to an aromatic hydrocarbon group included in Ar and to a hydrogen atom (H) shown in general formula (5).

Regarding specific examples of the polycarboxylic acid ester compound (5), specific examples of compounds represented by general formula (5) where each Ar is a 1-oxybenzen-4-yl group include compounds (5-1) to (5-6).

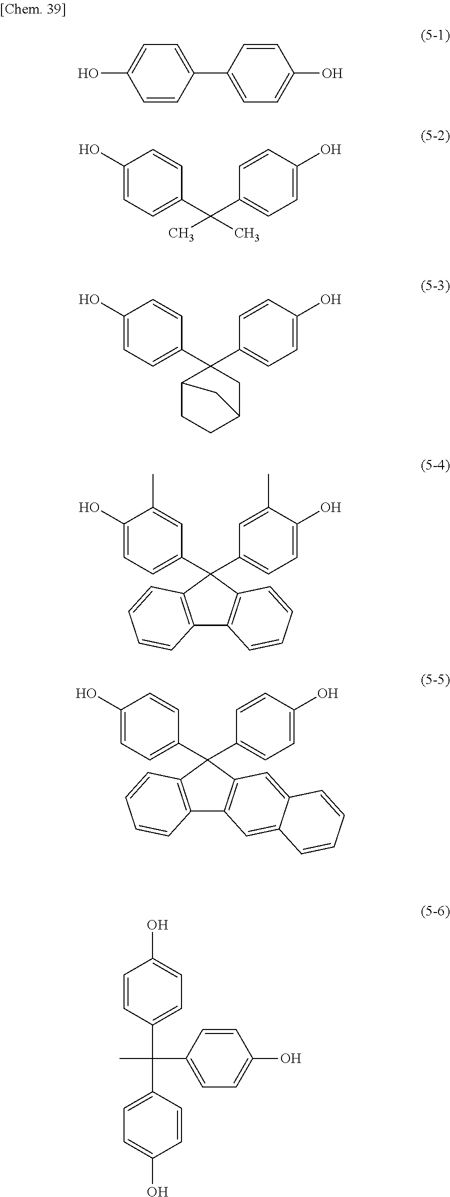

Specific examples of compounds represented by general formula (5) where each Ar is a 2-oxynaphthalen-1-yl group include compounds (5-7) and (5-8).

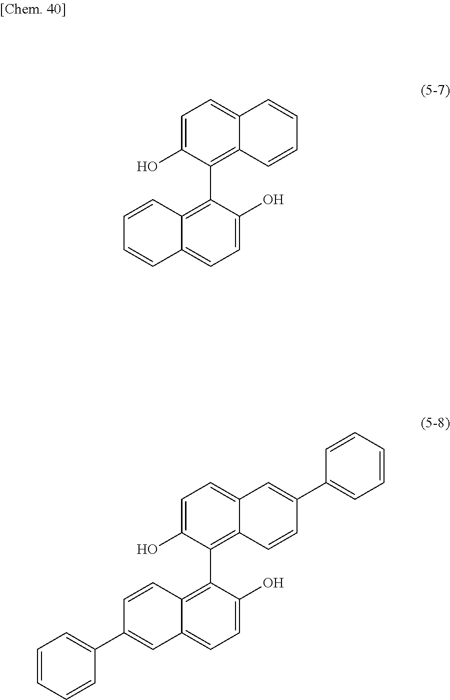

One specific example of a compound represented by general formula (5) where each Ar is a 2-oxynaphthalen-6-yl group is compound (5-9).

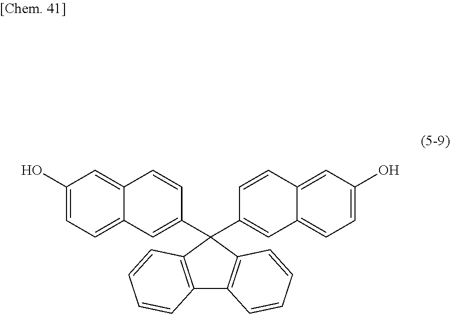

Specific examples of compounds represented by general formula (5) where each Ar is a 4-oxy-3-phenylbenzen-1-yl group include compounds (5-10) and (5-11).

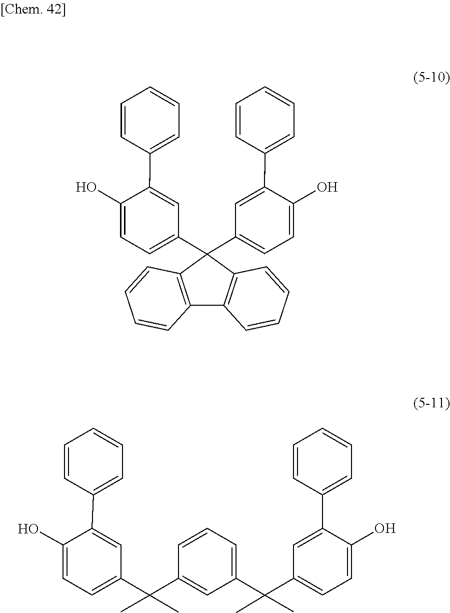

Specific examples of compounds represented by general formula (5) where each Ar is a 10-oxyphenanthren-9-yl group include compounds (5-12) and (5-13).

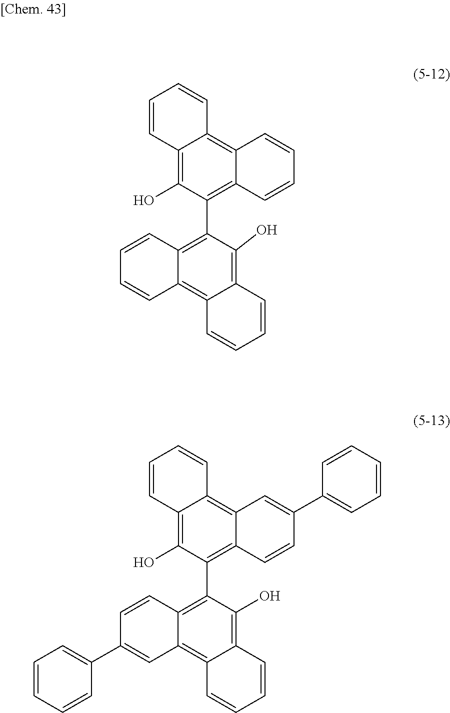

### <Halocarboxylic Acid Ester Compound Composition>

The halocarboxylic acid ester compound composition includes a halocarboxylic acid ester compound (6) represented by general formula (6) and a halocarboxylic acid ester compound (7) represented by general formula (7).

R₂ and R₃ in general formula (6) are as defined in general formula (1), and specific examples and preferred forms thereof are also the same.

Y in general formula (6) represents a halogen atom, and in particular, Y is preferably a chlorine atom or a bromine atom, particularly preferably a chlorine atom.

R₂ and R₄ in general formula (7) are as defined in general formula (2), and specific examples and preferred forms thereof are also the same.

Y in general formula (7) is as defined in general formula (6), and specific examples and preferred forms thereof are also the same.

Specific compounds of the halocarboxylic acid ester compound (6) and the halocarboxylic acid ester compound (7) are in common, and examples include alkyl haloacetates such as methyl chloroacetate, ethyl chloroacetate, n-propyl chloroacetate, isopropyl chloroacetate, n-butyl chloroacetate, isobutyl chloroacetate, tertiary butyl chloroacetate, methyl bromoacetate, ethyl bromoacetate, n-propyl bromoacetate, isopropyl bromoacetate, n-butyl bromoacetate, isobutyl bromoacetate, and tertiary butyl bromoacetate; alkenyl haloacetates such as vinyl chloroacetate, allyl chloroacetate, vinyl bromoacetate, and allyl bromoacetate; alkyl halopropanoates such as methyl 3-chloropropanoate, ethyl 3-chloropropanoate, methyl 3-bromopropanoate, and ethyl 3-bromopropanoate; and alkenyl halopropanoates such as vinyl 3-chloropropanoate, allyl 3-chloropropanoate, vinyl 3-bromopropanoate, and allyl 3-bromopropanoate.

Among them, an alkyl haloacetate or an alkenyl haloacetate is preferred, a compound selected from methyl chloroacetate, ethyl chloroacetate, methyl bromoacetate, ethyl bromoacetate, vinyl chloroacetate, and allyl chloroacetate is more preferred, a compound selected from methyl chloroacetate, ethyl chloroacetate, methyl bromoacetate, and ethyl bromoacetate is still more preferred, and methyl chloroacetate or ethyl chloroacetate is particularly preferred.

As the halocarboxylic acid ester compound (6), a compound in which the group represented by R₃ is the same as that of the polycarboxylic acid ester compound (1) to be produced is selected.

Particularly preferably, the halocarboxylic acid ester compound (6) is ethyl chloroacetate, and the halocarboxylic acid ester compound (7) is methyl chloroacetate.

In the halocarboxylic acid ester compound composition according to the production method of the present invention, the total component ratio of the halocarboxylic acid ester compound (6) and the halocarboxylic acid ester compound (7), as determined by gas chromatography analysis, is preferably 95 area % or more, more preferably 97 area % or more, still more preferably 99 area % or more, particularly preferably 99.5 area % or more, relative to the whole halocarboxylic acid ester compound composition.

In the halocarboxylic acid ester compound composition, the component ratio of the halocarboxylic acid ester compound (6) determined by gas chromatography analysis is not particularly limited, but is preferably 90 area % or more and 99.99 area % or less, more preferably 95 areas or more and 99.99 area % or less, still more preferably 97 area % or more and 99.99 area % or less, particularly preferably 98 area % or more and 99.99 areas or less.

In the halocarboxylic acid ester compound composition, the component ratio of the halocarboxylic acid ester compound (7) determined by gas chromatography analysis is not particularly limited, but is preferably 0.001 area % or more and 10 area % or less, more preferably 0.001 areas or more and 5 area % or less, still more preferably 0.001 areas or more and 1 area % or less, particularly preferably 0.001 area % or more and 0.1 area % or less.

The halocarboxylic acid ester compound composition may include, for example, a hydroxycarboxylic acid ester compound and an aliphatic alcohol as components other than the halocarboxylic acid ester compound (6) and the halocarboxylic acid ester compound (7).

The total component ratio of the halocarboxylic acid ester compound (6), the halocarboxylic acid ester compound (7), and other components, as determined by gas chromatography analysis of the halocarboxylic acid ester compound composition, is 100 area %.

### <Etherification Reaction Step>

The input molar ratio of the halocarboxylic acid ester compound (6) relative to the polyhydroxy aromatic compound (5) in the etherification reaction step according to the production method of the present invention is not particularly limited as long as it is more than or equal to the theoretical value (1.0+n), and the halocarboxylic acid ester compound (6) is used typically in the range of 2— to 20-fold molar amount, preferably in the range of 2— to 10-fold molar amount, more preferably in the range of 2— to 6-fold molar amount. Note that “n” of the theoretical value is the number of “n” in general formula (1).

The etherification reaction is carried out in the presence of a base, and examples of the base for use include triethylamine, pyridine, sodium hydroxide, potassium hydroxide, sodium carbonate, potassium carbonate, sodium hydrogencarbonate, and potassium hydrogencarbonate. Among them, sodium carbonate and potassium carbonate are preferred. The input molar ratio of the base relative to the total amount of the halocarboxylic acid ester compound (6) used is typically in the range of 0.8- to 4-fold molar amount, preferably in the range of 0.85- to 3-fold molar amount, more preferably in the range of 0.9- to 2-fold molar amount.

A catalyst may be used, and examples include alkali metal bromides such as sodium bromide and potassium bromide, alkali metal iodides such as sodium iodide and potassium iodide, ammonium bromide, and ammonium iodide. The amount of the catalyst used is typically in the range of 0.1 to 100 wt %, preferably in the range of 0.1 to 20 wt %, more preferably in the range of 0.1 to 10 wt %, relative to the amount of the polyhydroxy aromatic compound (5).

### (Etherification Reaction Temperature)

The reaction temperature is typically in the range of 25° C. to 120° C., preferably in the range of 40° C. to 100° C., more preferably in the range of 50° C. to 90° C., particularly preferably in the range of 60° C. to 80° C. A high reaction temperature leads to a low yield, and a low reaction temperature leads to a slow reaction rate, which are not preferred.

### (Etherification Reaction Pressure)

The reaction pressure is not limited, and may be normal pressure, reduced pressure, or increased pressure. Normal pressure or reduced pressure is preferred. In the case of a reaction under increased pressure, for example, the reaction can be carried out in a pressurized state under a stream of a gas inert to the reaction, such as nitrogen. That is, the reaction can be carried out while the inert gas is introduced into the reaction system and the gas in the reaction system is discharged. By doing so, carbonic acid gas produced from the carbonate or hydrogencarbonate used in the reaction can be discharged out of the reaction system, so that the reaction can be promoted. Specific examples of the gas inert to the reaction include nitrogen gas, argon gas, and helium gas, and nitrogen gas is most preferred from the economical viewpoint.

From the viewpoint of shortening the reaction time, reduced pressure is more preferred. By carrying out the reaction under reduced pressure, carbonic acid gas produced from the carbonate or hydrogencarbonate used in the reaction can be discharged out of the reaction system, so that the reaction is promoted, thus enabling a reaction time shorter than that of the reaction under normal pressure. Furthermore, by distilling the solvent out of the reaction system while carrying out the reaction under reduced pressure, the formation of by-products can be suppressed. Specifically, the reaction pressure is preferably in the range of 5 kPa or more and 80 kPa or less, more preferably in the range of 10 kPa or more and 70 kPa or less, still more preferably in the range of 30 kPa or more and 60 kPa or less. The reaction pressure can be brought to reduced pressure with a pressure reducing device, and when the reaction pressure is maintained in the above range, the pressure reducing device may be operated intermittently or continuously, and is preferably operated continuously. From the start to the end of the reaction, the reaction is preferably carried out under reduced pressure, specifically, while the pressure in the above range is maintained.

### (Etherification Reaction Solvent)

The reaction may be carried out without using a reaction solvent, but is preferably carried out using a reaction solvent for reasons of, for example, ease of operation in industrial production and improvement in reaction rate. The reaction solvent is not particularly limited as long as it does not distill out of a reaction vessel at a reaction temperature and is inert to the reaction, and examples include ketones such as acetone, methyl ethyl ketone, methyl isobutyl ketone, and cyclohexanone, ethers such as tetrahydrofuran, 1,4-dioxane, 1, 3-dioxane, and diethoxyethane, polar aprotic solvents such as acetonitrile, dimethylsulfoxide, dimethylformamide, and N-methylpyrrolidone, and aromatic hydrocarbon solvents such as toluene, xylene, and mesitylene. These organic solvents may each be used alone, or may be used in combination of two or more as appropriate to adjust polarity. Among them, the reaction solvent is preferably a C3 to C9 ketone solvent such as acetone, methyl ethyl ketone, methyl isobutyl ketone, cyclopentanone, cyclohexanone, cycloheptanone, or cyclooctanone or a polar aprotic solvent such as acetonitrile, dimethylsulfoxide, dimethylformamide, or N-methylpyrrolidone, more preferably a C3 to C9 ketone solvent or acetonitrile, still more preferably a C3 to C6 ketone solvent, particularly preferably acetone or methyl isobutyl ketone. Using methyl isobutyl ketone as the reaction solvent is preferred also because water washing for removing water-soluble impurities such as salts can be performed after completion of the reaction.

The solvent used in the etherification reaction is preferably dehydrated.

The amount of the solvent used is not particularly limited as long as the reaction is not hindered; typically, the amount of the solvent used is preferably in the range of 1 to 7 times by weight, more preferably in the range of 2 to 4 times by weight, still more preferably in the range of 2 to 3 times by weight, relative to the amount of the polyhydroxy aromatic compound (5).

When the reaction is carried out under reduced pressure while the solvent is distilled out of the reaction system, the amount of the solvent used is preferably in the range of 1.5 to 10 times by weight, more preferably in the range of 2 to 8 times by weight, still more preferably in the range of 2 to 6 times by weight, relative to the amount of the polyhydroxy aromatic compound (5).

The amount of distillation per hour during the period in which the reaction is carried out while the solvent is distilled out of the reaction system is preferably in the range of 0.05 to 1.5 times by weight, more preferably in the range of 0.1 to 1.0 parts by weight, still more preferably in the range of 0.3 to 1.0 times by weight, particularly preferably in the range of 0.3 to 0.8 times by weight, relative to the amount of the polyhydroxy aromatic compound (5). During the reaction, the amount of distillation per hour may vary within the above range, or the amount of distillation may temporarily exceed or fall below the upper or lower limit of the above range.

The amount of water in a reaction liquid in the etherification reaction step is preferably in the range of 0.01 wt % or more and 2.0 wt % or less relative to the amount of the polyhydroxy aromatic compound (5). When the amount of water in a reaction liquid is in this range, the polycarboxylic acid ester compound (1), which is the target compound of the production method of the present invention, can be produced in good reaction yield. The upper limit of the amount of water is more preferably in the range of 1.5 wt % or less, still more preferably in the range of 1.0 wt % or less, particularly preferably in the range of 0.5 wt % or less. Examples of methods for adjusting the amount of water in a reaction liquid to be within this range include using raw materials and solvents that have been dehydrated in advance, and removing water by distillation before performing the etherification reaction.

### (Etherification Reaction Endpoint)

The endpoint of the etherification reaction can be determined by liquid chromatography or gas chromatography analysis. The endpoint of the reaction is preferably defined as the time point at which the unreacted polyhydroxy aromatic compound (5) has disappeared and a mono-etherified product as an intermediate (in the case where n of the polycarboxylic residue ester compound (1), which is the target compound, is 2) or a bis-etherified product as an intermediate (in the case where n is 3) is hardly observed after being formed. Specifically, the time point at which the reaction intermediate is hardly observed after being formed is the time point at which the amount of the reaction intermediate determined by the above analysis has decreased to 1.5 area % or less, more preferably 1.0 area % or less, still more preferably 0.8 area % or less, particularly preferably 0.5 area % or less.

Although the reaction time varies depending on the reaction conditions such as reaction temperature, the reaction is typically completed in about 1 to 30 hours.

### <Aliphatic Alcohol (4) Represented by General Formula (4)>

R₃ in general formula (4) is as defined in general formula (1), and specific examples and preferred forms thereof are also the same.

Specific examples of the aliphatic alcohol (4) represented by general formula (4) include methanol, ethanol, 1-propanol, 2-propanol, 1-butanol, octyl alcohol, and allyl alcohol.

<Step of Reaction with Aliphatic Alcohol (4)>

### (Amount of Raw Material Used)

The weight of the aliphatic alcohol (4) used relative to the total weight of at least one selected from the group consisting of the polycarboxylic acid ester compound (2) and the polycarboxylic acid ester compound (3) is preferably in the range of 5 to 500 times by weight, more preferably in the range of 10 to 300 times by weight, still more preferably of in the range 10 to 200 times by weight, particularly preferably in the range of 15 to 150 times by weight.

(Solvent for Reaction with Aliphatic Alcohol (4))

In the step of reaction with the aliphatic alcohol (4), the reaction is typically carried out in the presence of a solvent. The solvent is not particularly limited as long as it does not inhibit the reaction, and it is preferable to use a polar aprotic solvent. Specific examples include chain aliphatic ketones having 3 to 9 carbon atoms, such as acetone (3 carbon atoms), methyl ethyl ketone (4 carbon atoms), diethyl ketone (5 carbon atoms), methyl isobutyl ketone (6 carbon atoms), methyl amyl ketone (7 carbon atoms), and methyl hexyl ketone (8 carbon atoms), chain nitrile solvents having 2 to 6 carbon atoms, such as acetonitrile and propanenitrile, ether solvents such as diethyl ether and tetrahydrofuran, and ester solvents such as ethyl acetate, dimethylformamide, and dimethylsulfoxide. Among them, the solvent is preferably a chain aliphatic ketone having 3 to 9 carbon atoms or acetonitrile, more preferably a chain aliphatic ketone having 3 to 9 carbon atoms, still more preferably a chain aliphatic ketone having 3 to 6 carbon atoms, particularly preferably acetone or methyl isobutyl ketone. Using methyl isobutyl ketone as the reaction solvent is preferred also because water washing for removing water-soluble impurities such as salts can be performed. These solvents may be used alone or as a mixture of two or more.

The solvent used for the reaction with the aliphatic alcohol (4) is preferably dehydrated.

The amount of the solvent used for the reaction is not particularly limited as long as there are no problems with operability and handleability, but is preferably in the range of 1.5 to 5 times by weight, more preferably in the range of 1.5 to 3.5 times by weight, still more preferably in the range of 1.5 to 2.5 times by weight, relative to the solids used for the reaction.

(Catalyst for Reaction with Aliphatic Alcohol (4))

In the step of reaction with the aliphatic alcohol (4), a catalyst for accelerating the reaction is not particularly necessary, but an acid catalyst or a base catalyst can be used as needed. In this case, examples of acid catalysts that can be used include, but are not limited to, concentrated hydrochloric acid, hydrochloric acid gas, trifluoroacetic acid, methanesulfonic acid, p-toluenesulfonic acid, benzoic acid, and mixtures thereof, and examples of base catalysts that can be used include, but are not limited to, sodium hydroxide, sodium carbonate, triethylamine, triethanolamine, and mixtures thereof.

(Temperature, Pressure, and Time of Reaction with Aliphatic Alcohol (4))

The reaction temperature in the step of reaction with the aliphatic alcohol (4) is a temperature lower than the boiling point of the aliphatic alcohol (4) for use. The limit thereof varies depending on the boiling point, and is preferably in the range of 30° C. to 100° C., more preferably in the range of 30° C. to 80° C., still more preferably in the range of 40° C. to 80° C., particularly preferably in the range of 40° C. to 60° C.

For the reaction pressure in the step of reaction with the aliphatic alcohol (4), the reaction may be carried out under normal pressure conditions, or may be carried out under increased pressure or reduced pressure.

The reaction time in the step of reaction with the aliphatic alcohol (4) varies depending on the reaction conditions such as reaction temperature, and the reaction is typically completed in about 1 to 48 hours.

### Other Embodiments of Production Method of Present Invention

Other embodiments of the production method of the present invention include the following embodiments.

### Embodiment 1

A method for producing a polycarboxylic acid ester compound (1), the method including an etherification reaction step of performing an etherification reaction using a polyhydroxy aromatic compound (5) and a halocarboxylic acid ester compound composition including a halocarboxylic acid ester compound (6) and a halocarboxylic acid ester compound (7) to obtain a polycarboxylic acid ester compound (2), and a step of reaction with an aliphatic alcohol (4), the step including reacting the polycarboxylic acid ester compound (2) obtained through the etherification step with the aliphatic alcohol (4) to obtain the carboxylic acid ester compound (1).

### Embodiment 2

A method for producing a polycarboxylic acid ester compound (1), the method including an etherification reaction step of performing an etherification reaction using a polyhydroxy aromatic compound (5) and a halocarboxylic acid ester compound (6) to obtain a polycarboxylic acid ester compound (3), and a step of reaction with an aliphatic alcohol (4), the step including reacting the polycarboxylic acid ester compound (3) obtained through the etherification step with the aliphatic alcohol (4) to obtain the carboxylic acid ester compound (1).

### Embodiment 3

A method for producing a polycarboxylic acid ester compound (1), the method including an etherification reaction step of performing an etherification reaction using a polyhydroxy aromatic compound (5) and a halocarboxylic acid ester compound composition including a halocarboxylic acid ester compound (6) and a halocarboxylic acid ester compound (7) to obtain a polycarboxylic acid ester compound (2) and a polycarboxylic acid ester compound (3), and a step of reaction with an aliphatic alcohol (4), the step including reacting the polycarboxylic acid ester compound (2) and the polycarboxylic acid ester compound (3) obtained through the etherification step with the aliphatic alcohol (4) to obtain the carboxylic acid ester compound (1).

In Embodiments 1 to 3, the polycarboxylic acid ester compound (1) represented by general formula (1) may be formed through the etherification reaction step, and is preferably formed.

### <Separation and Purification of Polycarboxylic Acid Ester Compound (1)>

From the final reaction mixture obtained through the step of reaction with the aliphatic alcohol (4), the polycarboxylic acid ester compound (1) can be obtained by performing separation and purification in accordance with a conventional method. For example, post-treatment operations such as water washing, crystallization, filtration, distillation, and separation by column chromatography or other means can be performed. For higher purity, distillation, recrystallization, or purification by column chromatography may be further performed in accordance with a conventional method.

The step of reaction with the aliphatic alcohol (4), the etherification reaction step, and any other steps such as mixing of raw materials, neutralization, distillation, crystallization, filtration, and drying in the present production method are preferably carried out in an atmosphere where the content of oxygen, which can cause oxidation degradation, coloration, and electrostatic ignition due to volatilized solvent, is low or in an inert gas atmosphere such as nitrogen or argon.

### EXAMPLES

The present invention will now be described more specifically with reference to Examples, but it should be noted that the present invention is not limited to these

Examples. Analysis methods are as follows.

### <Analysis Methods>

### 1. Composition Analysis of Polycarboxylic Acid Ester Compound

The composition analysis of polycarboxylic acid ester compounds was performed by high-performance liquid chromatography (HPLC) with the following apparatus and conditions. In the obtained analysis results, “%” means an area percentage.

Measurement apparatus: high-performance liquid chromatography analyzer (manufactured by Shimadzu Corporation)
Pump: LC-20AD
Column oven: CTO-20A
Detector: SPD-20A
Column: HALO-C18
Oven temperature: 50° C.
Flow rate: 0.7 mL/min.
Detection wavelength: 280 nm

### Gradient Conditions

Mobile phase: (A) 0.2 vol % aqueous acetic acid solution, (B) methanol
(B) vol % (time from start of analysis) 50% (0 min)→100% (10 min)→100% (13 min)

### 2. Composition Analysis of Halocarboxylic Acid Ester

Measurement apparatus: gas chromatography (GC)
Injection mode: split
Vaporization chamber temperature: 300.0° C.
Pressure: 164.0 kPa
Flow rate: 33.7 mL/min.
Column flow rate: 1.46 ml/min.
Split ratio: 20.0
Column: TC-1 0.25 mm×60 m
Detector: FID
Detector temperature: 300.0° C.
Temperature program: 40° C. (0 min)−40° C. (5 min)→250° C. (15.5 min)>250° C. (20.5 min)

### Comparative Example 1

Using a crystal of 2,2′-bis(2-ethoxycarbonylmethoxy)-1, 1′-binaphthyl analyzed by HPLC and found to contain 98.8% of 2, 2′-bis(2-ethoxycarbonylmethoxy)-1,1′-binaphthyl (compound (1-7)), 0.6% of compound (2-7), and 0.5% of compound (3-7), a four-necked flask was charged with 50.0 g of the crystal and 32.0 g of methyl isobutyl ketone and heated to 73° C. to dissolve the crystal. Thereafter, 32.0 g of normal heptane was added at this temperature, and cooling was started while stirring was continued. A seed crystal was added at 70° C., the temperature was held at 50° C. for 1 hour and then lowered to 25° C., and stirring was continued overnight. Thereafter, the crystal was separated by filtration. The obtained crystal was dried to obtain 47.4 g of a crystal of 2, 2′-bis(2-ethoxycarbonylmethoxy)-1, 1 ′-binaphthyl purified by recrystallization.

The obtained crystal was analyzed by HPLC and found to contain 99.0% of compound (1-7), 0.6% of compound (2-7), and 0.3% of compound (3-7).

The content ratio of compound (3-7) decreased by 0.2% as a result of purification by recrystallization, that is, a 40% decrease relative to the original content ratio was achieved, whereas the content ratio of compound (2-7) did not change, revealing that compound (2-7) cannot be removed by purification by recrystallization.

The only structural difference between compound (2-7) and 2, 2′-bis(2-ethoxycarbonylmethoxy)-1,1′-binaphthyl (compound (1-7)), which is the target compound, is that one ester group is ethyl or methyl, and their difference in solubility is not large. This is presumably why the impurity and the target could not be separated from each other by purification by recrystallization.

### Comparative Example 2

A four-necked flask was charged with 15.0 g (0.05 mol) of 1, 1′-binaphthalene-2, 2′-diol (compound (5-7)), 15.0 g of potassium carbonate, and 0.8 g of potassium iodide and purged with nitrogen, and then 22.5 g of super-dehydrated acetone was added. Thereafter, the temperature was raised to 60° C., and while this temperature was maintained, 16.1 g (0.13 mol) of ethyl chloroacetate (content ratio by GC analysis:ethyl chloroacetate 99.50 area %, methyl chloroacetate 0.05 area %) to which 0.1 g of N-methylpyrrolidone had been added was added dropwise over 2 hours. Thereafter, an etherification reaction was carried out for 19 hours while the temperature was maintained at 60° C.

The liquid after the etherification reaction was analyzed by HPLC and found to contain 98.0% of compound (1-7), 0.2% of compound (2-7), and 1.3% of compound (3-7).

Next, 60.0 g of water was added to the liquid after the etherification reaction containing compound (2-7) and compound (3-7) while the liquid temperature was maintained at 60° C., and then the mixture was cooled to 30° C. and stirred to precipitate a crystal. Thereafter, the crystal was separated by filtration.

The crystal separated by filtration was analyzed by HPLC and found to contain 98.5% of compound (1-7), 0.2% of compound (2-7), and 1.2% of compound (3-7).

A four-necked flask was charged with the crystal separated by filtration and 15.0 g of methyl isobutyl ketone to dissolve the crystal. A water-washing operation involving the addition of 15.0 g of water into the flask, stirring at 75° C. for 30 minutes, and the removal of an aqueous layer separated by standing was repeated three times. To an oil layer left after the washing with water, 21.0 g of normal heptane was added, and the mixture was cooled to 25° C. while stirring was continued. After stirring was continued overnight, a precipitated crystal was separated by filtration. The obtained crystal was dried to obtain 17.6 g (yield: 73%) of a crystal of compound (1-7).

The obtained crystal was analyzed by HPLC and found to contain 99.2% of compound (1-7), 0.2% of compound (2-7), and 0.6% of compound (3-7).

The hue of the obtained crystal of compound (1-7) determined with a 30 wt % tetrahydrofuran (THF) solution was 20 on APHA scale.

### Example 1

In Example 1, 2, 2′-bis(2-ethoxycarbonylmethoxy)-1,1′-binaphthyl (compound (1-7)) was produced by a method including a step of reaction with aliphatic alcohol after the etherification reaction step in the production method of Comparative Example 2.

A four-necked flask was charged with 25.0 g (0.09 mol) of compound (5-7), 25.0 g of potassium carbonate, and 1.3 g of potassium iodide and purged with nitrogen. Thereafter, 37.5 g of super-dehydrated acetone was added, and the temperature was raised to 60° C., after which while this temperature was maintained, 26.8 g (0.22 mol) of ethyl chloroacetate (content ratio by GC analysis:ethyl chloroacetate 99.50 area %, methyl chloroacetate 0.05 area %) to which 0.2 g of N-methylpyrrolidone had been added was added dropwise over 2 hours. Thereafter, an etherification reaction was carried out for 21 hours while the temperature was maintained at 60° C.

The reaction liquid obtained by the etherification reaction was analyzed by HPLC and found to contain 98.4% of compound (1-7), 0.2% of compound (2-7), and 0.9% of compound (3-7).

With the area percentage of each compound calculated by the HPLC analysis of the reaction liquid regarded as the weight ratio in the reaction liquid, and with the theoretical yield, 40.0 g, of compound (1-7) obtainable by the etherification reaction regarded as the total amount of components produced by the etherification reaction and detected by HPLC, the yields (g) of compound (2-7) and compound (3-7) from the etherification reaction were estimated. As a result, it is estimated that compound (2-7) is produced in an amount of 0.08 g (40 (g)×0.2(%)=0.08 (g)), and compound (3-7) is produced in an amount of 0.36 g (40 (g)×0.9(%)=0.36 (g)).

Thereafter, 48.0 g of super-dehydrated ethanol was added to the liquid after the etherification reaction containing compound (2-7) and compound (3-7), and the reaction with aliphatic alcohol was carried out by performing stirring for 6 hours while maintaining the liquid temperature at 50° C.

The reaction liquid obtained by the reaction with aliphatic alcohol was analyzed by HPLC, and it was found that as compared with before the reaction with aliphatic alcohol, the detected amounts (detected peak sizes) of compound (2-7) and compound (3-7) decreased after the reaction. Compound (1-7), 99.2%; compound (2-7), 0.1%; compound (3-7), less than 0.1%.

Next, 100.0 g of water was added to the liquid after the reaction with alcohol while the liquid temperature was maintained at 50° C., and then the mixture was cooled to 25° C. and stirred to precipitate a crystal. Thereafter, the crystal was separated by filtration.

A four-necked flask was charged with the crystal separated by filtration and 25.0 g of methyl isobutyl ketone to dissolve the crystal. A water-washing operation involving the addition of 25.0 g of water into the flask, stirring at 80° C. for 30 minutes, and the removal of an aqueous layer separated by standing was repeated three times.

To an oil layer left after the washing with water, 30.0 g of normal heptane was added, and the liquid was cooled to 25° C. while stirring was continued. After stirring was continued overnight, a precipitated crystal was separated by filtration. The obtained crystal was dried to obtain 35.7 g (yield: 89%) of a crystal of compound (1-7).

The obtained crystal was analyzed by HPLC and found to contain 99.9% of compound (1-7), 0.1% of compound (2-7), and less than 0.1% of compound (3-7).

The hue of the obtained crystal of compound (1-7) determined with a 30 wt % THE solution was 10 on APHA scale.

As described above, it has become clear that 2,2 ‘-bis(2-ethoxycarbonylmethoxy)-1, 1’-binaphthyl (compound (1-7)) having a low impurity content, high purity, and improved quality can be produced.

In Example 1, the reaction with aliphatic alcohol was performed. Before the reaction with aliphatic alcohol, the content ratios were as follows: compound (2-7), 0.2%; compound (3-7), 0.9%. After the reaction with aliphatic alcohol, the content ratios were reduced as follows: compound (2-7), 0.1%; compound (3-7), less than 0.1%.

In Example 1, as compared with Comparative Example 2, compound (2-7) contained in the obtained compound (1-7) decreased by 0.1%, and compound (3-7) decreased by 0.6% to be practically not contained. As a result, compound (1-7) having higher purity was obtained.

From this, it has become clear that according to the production method of the present invention, a specific impurity that cannot be removed or can only be removed with limited effectiveness by a recrystallization operation can be reduced, and compound (1-7) having further improved quality can be obtained.

### Example 2

A four-necked flask was charged with 35.0 g (0.12 mol) of compound (5-7), 35.5 g of potassium carbonate, and 1.8 g of potassium iodide and purged with nitrogen. Thereafter, 87.5 g of methyl isobutyl ketone was added, and then the temperature was raised to 92° C., after which 35.3 g of methyl isobutyl ketone was distilled out under a reduced pressure of 43 kPa. Thereafter, the pressure was returned to normal with nitrogen, and while the temperature was maintained, 37.5 g (0.31 mol) of ethyl chloroacetate (content ratio by GC analysis:ethyl chloroacetate 99.57 area %, methyl chloroacetate 0.10 area %) to which 0.3 g of N-methylpyrrolidone had been added was added dropwise over 2 hours. Thereafter, an etherification reaction was carried out for 13 hours while the temperature was maintained at 90° C.

The liquid obtained by the etherification reaction was analyzed by HPLC and found to contain 93.2% of compound (1-7), 0.7% of compound (2-7), and 4.0% of compound (3-7).

With the area percentage of each compound calculated by the HPLC analysis of the reaction liquid regarded as the heavy weight ratio in the reaction liquid, and with the theoretical yield, 56.0 g, of compound (1-7) obtainable by the etherification reaction regarded as the total amount of components produced by the etherification reaction and detected by HPLC, the yields (g) of compound (2-7) and compound (3-7) from the etherification reaction were estimated. As a result, it is estimated that compound (2-7) is produced in an amount of 0.39 g (56.0 (g)×0.7(%)≈0.39 (g)), and compound (3-7) is produced in an amount of 2.24 g (56.0 (g)×4.0(%)=2.24 (g)).

Thereafter, 34.0 g of super-dehydrated ethanol was added to the liquid after the etherification reaction containing compound (2-7) and compound (3-7), and stirring was performed for 3 hours while the liquid temperature was maintained at 65° C. to 70° C. Thereafter, 22.0 g of super-dehydrated ethanol was added again, and stirring was performed for 2 hours while the liquid temperature was maintained at 65° C. to 70° C.

The liquid after the reaction with aliphatic alcohol was analyzed by HPLC, and it was found that as compared with before the reaction with aliphatic alcohol, the detected amounts (detected peak sizes) of compound (2-7) and compound (3-7) decreased after the reaction. Compound (1-7), 97. 4%; compound (2-7), 0.2%; compound (3-7), 0.2%.

Next, 140.0 g of water was added to the liquid after the reaction with aliphatic alcohol while the liquid temperature was maintained at 76° C., and then an oil layer was washed with water to remove an aqueous layer. While the liquid temperature was maintained at 76° C., 17.5 g of water was added and stirred, after which an aqueous layer was removed in the same manner as the above operation.

To an oil layer with a liquid temperature of 76° C. left after the washing with water, 52.5 g of normal heptane was added, and after the liquid was cooled while stirring was continued, a seed crystal was added to the liquid at 60° C. A crystal precipitated in a large amount at 52° C. Thereafter, cooling and stirring were continued until 25° C. was reached, and then the precipitated crystal was separated by filtration.

The crystal separated by filtration was analyzed by HPLC and found to contain 99.5% of compound (1-7), 0.2% of compound (2-7), and 0.1% of compound (3-7).

A four-necked flask was charged with the crystal obtained by separation by filtration and 28.0 g of methyl isobutyl ketone and purged with nitrogen. Thereafter, the temperature was raised to 78° C. to dissolve the crystal. A water-washing operation involving the addition of 18.0 g of water into the flask, stirring at 80° C. for 30 minutes, and the removal of an aqueous layer separated by standing was repeated twice. To an oil layer left after the washing with water, 35.0 g of normal heptane was added, and cooling of the liquid was started while stirring was continued. A seed crystal was added at 70° C., and the liquid was cooled to finally reach 25° C. A precipitated crystal was separated by filtration, and the obtained crystal was dried to obtain 47.0 g (yield: 83.9%) of a crystal of compound (1-7).

The obtained crystal was analyzed by HPLC and found to contain 99.8% of compound (1-7), 0.2% of compound (2-7), and less than 0.1% of compound (3-7).

The hue of the obtained crystal of compound (1-7) determined with a 30 wt % tetrahydrofuran (THF) solution was 40 on APHA scale.

### Example 3

A four-necked flask was charged with 300.4 g (0.78 mol) of 9, 9′-biphenanthrene-10, 10′-diol (compound (5-12)), 225. 4 g (1.63 mol) of potassium carbonate, and 15.1 g (0.09 mol) of potassium iodide and purged with nitrogen. Thereafter, 798 g of super-dehydrated acetone was loaded, the temperature was raised to 55° C., and 239.1 g (1.95 mol) of ethyl chloroacetate (content ratio by GC analysis:ethyl chloroacetate 99.63 area %, methyl chloroacetate 0.01 area %) was added dropwise over 2 hours. After the dropwise addition, an etherification reaction was carried out by continuing stirring for 14 hours while maintaining the temperature at 55° C.

The reaction liquid obtained by the etherification reaction was analyzed by HPLC and found to contain 98.3% of 10, 10′-bis(2-ethoxycarbonylmethoxy)-9, 9′-biphenantoyl (compound (1-12)), 0.3% of compound (2-12), and 0.7% of compound (3-12).

With the area percentage of each compound calculated by the HPLC analysis of the reaction liquid regarded as the heavy weight ratio in the reaction liquid, and with the theoretical yield, 434.5 g, of compound (1-12) obtainable by the etherification reaction regarded as the total amount of components produced by the etherification reaction and detected by HPLC, the yields (g) of compound (2-12) and compound (3-12) from the etherification reaction were estimated. As a result, it is estimated that compound (2-12) is produced in an amount of 1.30 g (434.5 (g)×0.3(%)≈1.30 (g)), and compound (3-12) is produced in an amount of 3.04 g (434.5 (g)×0.7(%)≈3.04 (g)).

To the liquid containing compound (2-12) and compound (3-12), 431.3 g (9.36 mol) of super-dehydrated ethanol was added, and a reaction with aliphatic alcohol was carried out by continuing stirring for 2 hours while maintaining the liquid temperature at 50° C.

The reaction liquid obtained by the reaction with aliphatic alcohol was analyzed by HPLC, and it was found that as compared with before the reaction with aliphatic alcohol, after the reaction, the detected amounts (detected peak sizes) of compound (2-12) and compound (3-12) decreased, and the detected amount of compound (1-12), the target, increased. Compound (1-12), 99.2%; compound (2-12), 0.1%; compound (3-12), 0.1%.

Thereafter, the liquid after the reaction with aliphatic alcohol was cooled and continuously stirred at 25° C. overnight, and then a precipitated crystal was separated by filtration.

A four-necked flask was charged with the crystal separated by filtration, 1358 g of methyl isobutyl ketone, and 900 g of water, purged with nitrogen, and heated to dissolve the crystal. Thereafter, an oil layer was washed with water at 80° C., and an aqueous layer was extracted. The water-washing operation of the oil layer was repeated four more times.

To the oil layer left after the washing with water, 600 g of normal heptane was added, and the liquid was cooled to 25° C. while stirring was continued. After stirring was continued overnight, a precipitated crystal was separated by filtration. The crystal separated by filtration was dried at 80° C. under reduced pressure to obtain 356.3 g (yield: 82%) of a crystal of compound (1-12).

The obtained crystal was analyzed by HPLC and found to contain 99.6% of compound (1-12), 0.1% of compound (2-12), and 0.1% of compound (3-12).

### Comparative Example 3

A four-necked flask was charged with 30.1 g (0.08 mol) of compound (5-12), 22.6 g (0.16 mol) of potassium carbonate, and 1.7 g (0.01 mol) of potassium iodide and purged with nitrogen. Thereafter, 75.0 g of methyl isobutyl ketone was added, and then the temperature was raised to 95° C., after which 28.0 g of methyl isobutyl ketone was distilled out under a reduced pressure of 50 kPa. Thereafter, the pressure was returned to normal with nitrogen, and while the temperature was maintained, 24.0 g (0.20 mol) of ethyl chloroacetate (content ratio by GC analysis:ethyl chloroacetate 99.57 area %, methyl chloroacetate 0.10 area %) was added dropwise over 2 hours. Thereafter, an etherification reaction was carried out for 10 hours while the temperature was maintained at 90° C.

The reaction liquid obtained by the etherification reaction was analyzed by HPLC and found to contain 96.0% of compound (1-12), 0.5% of compound (2-12), and 0.8% of compound (3-12).

Thereafter, without performing the step of reaction with aliphatic alcohol, 120 g of water and 45 g of methyl isobutyl ketone were added to the liquid after the etherification reaction. An oil layer was washed with water at 80° C., and an aqueous layer was extracted. The water-washing operation of the oil layer was repeated one more time. Thereafter, 92 g of normal heptane was added to the oil layer left after the washing with water, and the liquid was cooled to 25° C. while sales expansion was continued. After stirring was continued overnight, a precipitated crystal was separated by filtration.

The crystal separated by filtration was analyzed by HPLC and found to contain 98.9% of compound (1-12), 0.5% of compound (2-12), and 0.4% of compound (3-12).

A four-necked flask was charged with the crystal separated by filtration, 184 g of methyl isobutyl ketone, and 100 g of water, purged with nitrogen, and heated to dissolve the crystal. Thereafter, an oil layer was washed with water at 80° C., and an aqueous layer was extracted. The water-washing operation of the oil layer was repeated one more time. To the oil layer left after the washing with water, 107 g of normal heptane was added, and the liquid was cooled to 25° C. and continuously stirred overnight, after which a precipitated crystal was separated by filtration. The crystal separated by filtration was dried at 80° C. under reduced pressure to obtain 33.8 g (yield: 78%) of a crystal of compound (1-12).

The obtained crystal was analyzed by HPLC and found to contain 99.0% of compound (1-12), 0.5% of compound (2-12), and 0.4% of compound (3-12).

From the above, it has become clear that according to the production method of the present invention, a specific impurity that is contained in the polycarboxylic acid ester compound (1) and that cannot be removed or can only be removed with limited effectiveness by a crystallization operation or a recrystallization operation can be reduced, and thus the polycarboxylic acid ester compound (1) having improved quality can be produced.

1. A method for producing a polycarboxylic acid ester compound (1) represented by general formula (1), the method comprising a step of reaction with an aliphatic alcohol (4), the step including reacting at least one selected from the group consisting of a polycarboxylic acid ester compound (2) represented by general formula (2) and a polycarboxylic acid ester compound (3) represented by general formula (3) with the aliphatic alcohol (4) represented by general formula (4) to obtain the polycarboxylic acid ester compound (1) represented by general formula (1): 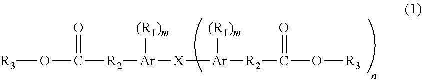
wherein each Ar independently represents a “(2+m)-valent” monooxy aromatic hydrocarbon group having 6 to 20 carbon atoms, each R₁ independently represents a linear or branched alkyl group having 1 to 6 carbon atoms or a cyclic alkyl group having 5 to 6 carbon atoms, each R₂ independently represents a linear or branched alkylene group having 1 to 4 carbon atoms, R₃ represents a linear or branched alkyl group having 1 to 10 carbon atoms or an alkenyl group having 2 to 10 carbon atoms, each m independently represents 0, 1, or 2, n represents 1 or 2, and X represents a single bond, an oxygen atom, a sulfur atom, a sulfonyl group, a carbonyl group, a divalent group represented by general formula (1a), (1b), or (1c), or a trivalent group represented by general formula (1d) or (1e); in general formula (1), an oxygen atom of Ar is bonded to an aromatic hydrocarbon group included in Ar and to R₂: 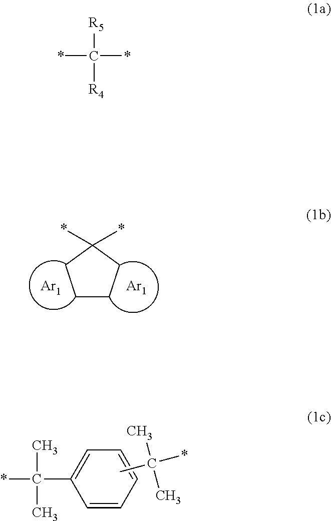
wherein, in general formula (1a), R₈ and R₆ each independently represent a hydrogen atom, an alkyl group having 1 to 10 carbon atoms, an alkyl halide group having 1 to 10 carbon atoms, or an aryl group having 6 to 12 carbon atoms, R₈ and R₆ may be bonded to each other to together form a cycloalkylidene group having 5 to 20 carbon atoms, and in general formula (1b), each Ar₁ independently represents an aryl group having 6 to 12 carbon atoms; each * in general formulas (1a), (1b), and (1c) represents a bonding position: 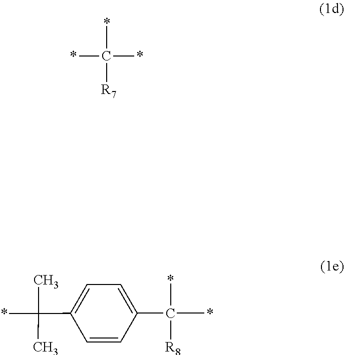
wherein, in general formula (1d), R₇ represents a hydrogen atom, an alkyl group having 1 to 10 carbon atoms, or an aryl group having 6 to 12 carbon atoms; in general formula (1e), R₈ represents a hydrogen atom or an alkyl group having 1 to 10 carbon atoms. Each * in general formulas (1d) and (1e) represents a bonding position: 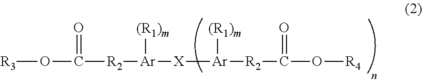
wherein Ar, R₁, R₂, R₃, m, n, and X in general formula (2) are as defined in general formula (1), and each R₄ independently represents a linear or branched alkyl group having 1 to 10 carbon atoms or an alkenyl group having 2 to 10 carbon atoms that is different from R₃: 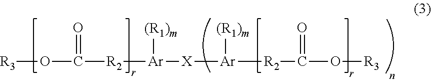
wherein Ar, R₁, R₂, m, n, X, and R₃ in general formula (3) are as defined in general formula (1), and each r independently represents an integer of 1 to 4. At least one r is 2, 3, or 4:
HO—R₃ (4)
wherein R₃ in general formula (4) is as defined in general formula (1).

2. The production method according to claim 1, wherein the polycarboxylic acid ester compound (2) represented by general formula (2) and the polycarboxylic acid ester compound (3) represented by general formula (3) are compounds obtained through an etherification reaction step of performing an etherification reaction using a polyhydroxy aromatic compound (5) represented by general formula (5) and a halocarboxylic acid ester compound composition including a halocarboxylic acid ester compound (6) represented by general formula (6) and a halocarboxylic acid ester compound (7) represented by general formula (7): 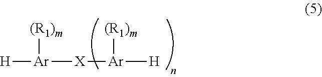
wherein Ar, R₁, m, n, and X in general formula (5) are as defined in general formula (1). In general formula (5), an oxygen atom of Ar is bonded to an aromatic hydrocarbon group included in Ar and to a hydrogen atom (H) shown in general formula (5): 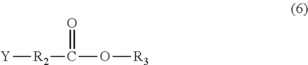
wherein R₂ and R₃ in general formula (6) are as defined in general formula (1), and Y represents a halogen atom: 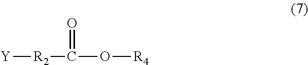
wherein R₂ and R₄ in general formula (7) are as defined in general formula (2), and Y is as defined in general formula (6).

3. The production method according to claim 1, wherein the polycarboxylic acid ester compound (2) is a compound obtained through an etherification reaction step of performing an etherification reaction using a polyhydroxy aromatic compound (5) represented by general formula (5) and a halocarboxylic acid ester compound composition including a halocarboxylic acid ester compound (6) represented by general formula (6) and a halocarboxylic acid ester compound (7) represented by general formula (7): 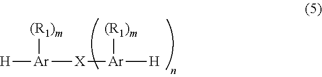
wherein Ar, R₁, m, n, and X in general formula (5) are as defined in general formula (1); in general formula (5), an oxygen atom of Ar is bonded to an aromatic hydrocarbon group included in Ar and to a hydrogen atom (H) shown in general formula (5): 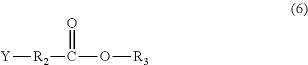
wherein R₂ and R₃ in general formula (6) are as defined in general formula (1), and Y represents a halogen atom: 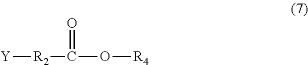
wherein R₂ and R₄ in general formula (7) are as defined in general formula (2), and Y is as defined in general formula (6).

4. The production method according to claim 1, wherein the polycarboxylic acid ester compound (3) is a compound obtained through an etherification reaction step of performing an etherification reaction using a polyhydroxy aromatic compound (5) represented by general formula (5) and a halocarboxylic acid ester compound (6) represented by general formula (6); 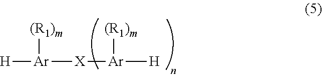
wherein Ar, R₁, m, n, and X in general formula (5) are as defined in general formula (1); in general formula (5), an oxygen atom of Ar is bonded to an aromatic hydrocarbon group included in Ar and to a hydrogen atom (H) shown in general formula (5): 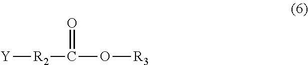
wherein R₂ and R₃ in general formula (6) are as defined in general formula (1), and Y represents a halogen atom.

5. The production method according to claim 1, wherein Ar is any one group selected from the group consisting of a 1-oxybenzen-4-yl group, a 1-oxybenzen-3-yl group, a 1-oxybenzen-2-yl group, a 1-oxynaphthalen-2-yl group, a 1-oxynaphthalen-4-yl group, a 1-oxynaphthalen-5-yl group, a 2-oxynaphthalen-1-yl group, a 2-oxynaphthalen-6-yl group, a 2-oxynaphthalen-7-yl group, a 4-oxy-3-phenylbenzen-1-yl group, a 9-oxyphenanthren-3-yl group, a 10-oxyphenanthren-9-yl group, a 2-oxyanthracen-7-yl group, a 1-oxy-3-phenylnaphthalen-4-yl group, a 1-oxy-3-phenylnaphthalen-5-yl group, a 2-oxy-1-phenylnaphthalen-6-yl group, a 2-oxy-1-phenylnaphthalen-7-yl group, a 2-oxy-3-phenylnaphthalen-6-yl group, a 2-oxy-3-phenylnaphthalen-7-yl group, a 4-oxy-3-(1-naphthyl)benzen-1-yl group, a 4-oxy-3-(2-naphthyl)benzen-1-yl group, a 4-oxy-3,5-diphenylbenzen-1-yl group, a 4-oxy-2-phenylphenanthren-6-yl group, a 4-oxy-2-phenylphenanthren-7-yl group, a 4-oxy-2-phenylphenanthren-8-yl group, a 4-oxy-2-phenylphenanthren-9-yl group, a 4-oxy-2-phenylphenanthren-10-yl group, and a 2-oxy-3-phenylanthracen-7-yl group.

6. The production method according to claim 5, wherein n is 2, and X is a direct bond.

7. The production method according to claim 6, wherein R₃ is a linear or branched alkyl group having 1 to 10 carbon atoms.
